(19) United States
(12) Patent Application Publication
(10) Pub. No.: US 2011/0256557 A1
(43) Pub. Date: Oct. 20, 2011

(54) IDENTIFYING PARATHYROID HORMONE AGONISTS AND ANTAGONISTS

(75) Inventors: Xu Cao, Baltimore, MD (US); Mei Wan, Baltimore, MD (US); Erik Mills Schwiebert, Birmingham, AL (US)

(73) Assignees: DISCOVERYBIOMED, INC., Birmingham, AL (US); THE UAB RESEARCH FOUNDATION, Birmingham, AL (US)

(21) Appl. No.: 13/127,001

(22) PCT Filed: Oct. 30, 2009
(86) PCT No.: PCT/US09/62818

§ 371 Date: Jul. 1, 2011

Related U.S. Application Data
(60) Provisional application No. 61/110,192, filed on Oct. 31, 2008.

(51) Int. Cl. G01N 33/566

(52) U.S. Cl. 435/7.23

(57) ABSTRACT

Provided herein are methods of screening for an agent that is a PTH agonist or antagonist. For example, provided is a method of screening for an agent that is a PTH agonist or antagonist, the method comprising contacting a cell with LRP6 and the agent to be screened, wherein the cell comprises a PTH1R, and determining the level of LRP6 binding to the PTH1R. An increased level of LRP6 binding to the PTH1R compared to a control indicates the agent is a PTH agonist. A decreased level of LRP6 binding to the PTH1R compared to a control indicates the agent is a PTH antagonist. Also provided are methods of treating a skeletal disorder in a subject, wherein the skeletal disorder is characterized by proliferative bone growth or reduced bone density.

### CROSS-REFERENCE TO RELATED APPLICATION

This application claims the benefit of U.S. Provisional Application No. 61/110,192, filed Oct. 31, 2008.

### STATEMENT REGARDING FEDERALLY FUNDED RESEARCH

This invention was made with government support under Grant Nos. R1DK057501 and RAR053973 awarded by the National Institutes of Health. The government has certain rights in the invention.

### BACKGROUND

Parathyroid hormone (PTH) is a circulating hormone that acts as the central regulator of calcium metabolism by directly targeting bone, kidney, and intestine. The classical concept of PTH action is that it regulates serum calcium levels by stimulating bone resorption; however, intermittent administration of PTH selectively stimulates bone formation. Significant progress has been made in determining PTH downstream signaling events. PTH binds to its receptor PTH1R and activates the G protein α subunits Gαₛ and Gα_{q}. This leads to the production of 3′,5′-cyclic adenosine-5′-monophosphate (cAMP) and activation of phospholipase C (PLC), which eventually results in the activation of protein kinase A (PKA) and protein kinase C (PKC). Activation of PKA is believed to mediate the anabolic effect of PTH on bone; however, the precise molecular mechanisms by which PKA mediates PTH responses in osteoblasts remain unresolved. In spite of the knowledge of the PTH downstream signaling events, PTH is still the only FDA-approved anabolic therapy for bone.

### SUMMARY

Provided are methods of screening for an agent that is a parathyroid hormone (PTH) agonist. Specifically, the method comprises contacting a cell with lipoprotein related protein 6 (LRP6) and the agent to be screened. The contacted cell comprises a parathyroid hormone 1 receptor (PTH1R), and the method further comprises determining the level of LRP6 binding to the PTH1R. An increased level of LRP6 binding to the PTH compared to a control indicates the agent is a PTH agonist.

Optionally, the methods comprise contacting a cell with a parathyroid hormone (PTH) polypeptide or a receptor-binding fragment thereof, a WNT polypeptide, and the agent to be screened. The cell comprises a parathyroid hormone 1 receptor (PTH1R) and a nucleotide sequence encoding a reporter protein operably linked to an inducible promoter, wherein the inducible promoter is activated by PTH1R. The level of reporter protein expression is determined. An increase in the level of reporter protein expression as compared to a control indicates the agent is a PTH agonist.

Also provided are methods of screening for an agent that is a PTH antagonist. Specifically, the method comprises contacting a cell with LRP6 and the agent to be screened. The contacted cell comprises PTH1R, and the method further comprises determining the level of LRP6 binding to the PTH1R. A decreased level of LRP6 binding to the PTH1R compared to a control indicates the agent is a PTH antagonist.

Optionally, the methods comprise contacting a cell with a parathyroid hormone (PTH) polypeptide or a receptor-binding fragment thereof, a WNT polypeptide, and the agent to be screened. The cell comprises a parathyroid hormone 1 receptor (PTH1R) and a nucleotide sequence encoding a reporter protein operably linked to an inducible promoter, wherein the inducible promoter is activated by PTH1R. The level of reporter protein expression is determined. A decrease in the level of reporter protein expression as compared to a control indicates the agent is a PTH antagonist.

Also provided are methods of treating or preventing skeletal disorders in a subject, wherein the skeletal disorder is characterized by proliferative bone growth as compared to a control. The method comprises identifying a subject with or at risk of developing the skeletal disorder, and administering to the subject an agent that inhibits the binding of LRP6 to PTH1R. The agent can, for example, be the agent identified in the screen for PTH antagonists.

Further provided are methods of treating or preventing a skeletal disorder in a subject, wherein the skeletal disorder is characterized by reduced bone density. The method comprises identifying a subject with or at risk of developing the skeletal disorder, and administering to the subject an agent that stimulates the binding of LRP6 to PTH1R. The agent can, for example, be the agent identified in the screen for PTH agonists.

### DESCRIPTION OF DRAWINGS

FIG. 1 shows a histogram demonstrating that PTH-stimulates a luciferase reporter with TCF/LEF-binding elements (TCF4-Luc) in UMR-106 cells. (*) P<0.01, n=3.

FIG. 2 shows a Western blot demonstrating that PTH induced stabilization of β-catenin in UMR-106 cells.

FIG. 3 shows a Western blot demonstrating that PTH induced stabilization of β-catenin in mouse primary preosteoblasts.

FIG. 4 shows Western blots demonstrating that PTH induced stabilization of β-catenin in HEK293 cells. The top panel shows the stabilization of β-catenin in HEK293 cells treated with PTH for increasing amounts of time. The bottom panel shows the stabilization of β-catenin in HEK293 cells treated with increasing concentrations of PTH.

FIG. 5 shows a Western blot demonstrating that PTH induced β-catenin stabilization is not affected by Fz8CRD.

FIG. 6 shows immunohistochemical images demonstrating the β-catenin levels in femur sections from 5 month old rats at the indicated time point post administration of PTH.

FIG. 7 shows a histogram demonstrating the quantification of β-catenin positive osteoblasts in the immunohistochemical images of FIG. 6. (*) P<0.005, (**) P<0.001 (in comparison with control), n=6.

FIG. 8 shows immunohistochemical images demonstrating the β-catenin levels in tibia sections from 2-month old male mice at the indicated time points post administration of PTH (top panel). The bottom panel shows a histogram demonstrating the quantification of β-catenin positive osteoblasts in the immunohistochemical images of the top panel. (*) P<0.005, (**) P<0.001 (in comparison with control), n=6.

FIG. 9 shows a Western blot demonstrating that LRP6-specific siRNA reduced the amount of LRP6 protein in HEK293 cells.

FIG. 10 shows a Western blot demonstrating that LRP6-specific siRNA reduced PTH-induced β-catenin stabilization in HEK293 cells.

FIG. 11 shows a histogram demonstrating that LRP-specific siRNA reduced PTH-stimulated TCF/LEF transcriptional activity in UMR-106 cells. (*) P<0.01 in comparison with control), n=3; (n.s.) not significant (in comparison with control), n=3.

FIG. 12 shows a histogram demonstrating that LRP6-specific siRNA reduced PTH-stimulated Osteocalcin gene expression in C2C12 cells as analyzed by quantitative real-time polymerase chain reaction (qRT-PCR) assay.

FIG. 13 shows a histogram demonstrating that LRP6-specific siRNA reduced PTH-stimulated RANKL gene expression in C2C12 cells as analyzed by qRT-PCR assay.

FIG. 14 shows a Western blot demonstrating the co-immunoprecipitation (co-IP) of endogenous LRP6 and endogenous PTH1R in UMR-106 cells. Binding of LRP6 and PTH1R increases in a time-dependent manner post PTH administration.

FIG. 15 shows a Western blot demonstrating that PTH enhances binding of exongenous PTH1R to exogenous LRP6 but not exogenous LRP5 in HEK293 cells.

FIG. 16 shows a Western blot demonstrating that LRP6, PTH1R, and PTH form a ternary complex in HEK293 cells.

FIG. 17 shows a model demonstrating the photobleaching-based fluorescence resonance energy transfer (FRET) assay for CFP and YFP fused to the C-terminus of PTH1R and LRP6, respectively.

FIG. 18 shows representative confocal images demonstrating the association of CFP-PTH1R with YFP-LRP6 at 5 minutes post PTH treatment in HEK293 cells.

FIG. 19 shows a histogram demonstrating the FRET efficiencies before and after photobleaching in the absence or presence of PTH. (*) P<0.001 (in comparison with unbleached), n=6; (n.s.) not significant (in comparison with unbleached).

FIG. 20 shows a histogram demonstrating that ventral injection of PTH and PTH1R RNA promotes LRP6-induced axis duplication in Xenopus.

FIG. 21 shows representative images demonstrating that ventral injection of PTH and PTH1R RNA promotes LRP6-induced axis duplication in Xenopus.

FIG. 22 shows a Western blot demonstrating that the N-terminal domain of LRP6 interacts with PTH1R in HEK293 cells.

FIG. 23 shows a histogram demonstrating the quantification of surface binding rates, i.e., the ratios of the number of cells showing green to the number of cells showing red.

FIG. 24 shows a Western blot demonstrating that soluble LRP6N disrupts binding of endogenous LRP6 with PTH1R in UMR-106 cells.

FIG. 25 shows a histogram demonstrating that soluble LRP6N inhibits PTH-induced TCF4/LEF luciferase activation in UMR-106 cells. (*) P<0.01 (in comparison with control), n=3; (n.s.) not significant (in comparison with control), n=3.

FIG. 26 shows a histogram demonstrating that soluble LRP6N does not inhibit LiCl-induced TCF4/LEF luciferase activation in UMR-106 cells. (*) P<0.01 (in comparison with control), n=3; (n.s.) not significant (in comparison with control), n=3.

FIG. 27 shows a Western blot demonstrating DKK1 reduced Wnt3a or PTH-induced β-catenin stabilization in HEK293 cells.

FIG. 28 shows a histogram demonstrating that DKK1 and Sclerostin inhibit PTH-induced TCF4/LEF luciferase activation in UMR-106 cells.

FIG. 29 shows a Western blot demonstrating that PTH-induces phosphorylation of endogenous LRP6 in UMR-106 cells.

FIG. 30 shows a Western blot demonstrating that PTH-induces axin1 recruitment to the cell membrane in mouse primary preosteoblasts.

FIG. 31 shows a Western blot demonstrating that LRP6 binds axin1 in HEK293 cells treated with PTH.

FIG. 32 shows a Western blot demonstrating that Fz8CRD does not inhibit PTH-induced LRP6 phosphorylation in HEK293 cells.

FIG. 33 shows a Western blot demonstrating that soluble LRP6N inhibits PTH-induced endogenous LRP6 phosphorylation.

FIG. 34 shows immunohistochemical images demonstrating the phosphorylated LRP6 levels in femur sections from 5-month old rats at the indicated time points post PTH administration.

FIG. 35 shows a histogram demonstrating the quantification of phosphorylated LRP6-positive osteoblasts in the immunohistochemical images of the FIG. 34. (*) P<0.005, (**) P<0.001 (in comparison with control), n=6.

FIG. 36 shows a histogram demonstrating that PTH C-terminal truncations fail to stimulate TCF4/LEF luciferase activity in UMR-106 cells.

FIG. 37 shows a Western blot demonstrating that PTH C-terminal truncations fail to stabilize β-catenin in UMR-106 cells.

FIG. 38 shows a Western blot demonstrating that PTH C-terminal truncations disrupt the binding of LRP6 with axin.

FIG. 39 shows a Western blot demonstrating that the PKA inhibitor, PKI (14-22), inhibits PTH-induced LRP6 phosphorylation.

FIG. 40 shows a Western blot demonstrating that PKA inhibitors, PKI (14-22) and H89, inhibit binding of LRP6 and axin in cells treated with PTH.

FIG. 41 shows a Western blot demonstrating the PKA inhibitor, H89, inhibits PTH-induced β-catenin stabilization but does not inhibit Wnt3a-induced β-catenin stabilization in UMR-106 cells.

FIG. 42 shows a histogram demonstrating PKA inhibitors, PKI (14-22) and H89, inhibit PTH-induced TCF4/LEF luciferase activity in UMR-106 cells.

FIG. 43 shows a Western blot demonstrating the PKA inhibitor, H89, does not inhibit Wnt3a-induced LRP6 phosphorylation.

FIG. 44 shows a histogram demonstrating PKA inhibitors, PKI (14-22) and H89, do not inhibit Wnt3a-induced TCF4/LEF luciferase activity in UMR-106 cells.

FIG. 45 shows a schematic of a high-throughput screen designed to find PTH agonists and antagonists. The cells were transfected with the TCF/LEF luciferase reporter. PTH treatment alone did not activate the luciferase reporter; however, PTH potentiated WNT stimulation of the luciferase reporter.

FIG. 46 shows a design of an assay to validate the high-throughput screen. FIG. 46A shows a 96-well plate. The upper left hand quadrant was treated with vehicle, the upper right hand quadrant was treated with PTH alone, the lower left hand quadrant was treated with WNT3a alone, and the lower right hand quadrant was treated with PTH and WNT3a. FIG. 46B shows a graph demonstrating that WNT3a treatment alone activates the luciferase reporter, and PTH and WNT3a treatment results in a synergistic stimulation of the luciferase reporter.

FIG. 47 shows a schematic of a 96-well high-throughput screen to identify small molecules that inhibit or enhance the synergistic stimulation of the TCF/LEF luciferase reporter by treatment of the cells with PTH and WNT3a.

FIG. 48 shows a schematic of an AlphaLISA assay to determine the levels of osteosclerostin secreted into the media after the cells are treated with PTH and WNT3a.

FIG. 49 shows a schematic of a secondary validation FRET bioassay for the WNT and PTH receptor complex. The assay is designed to determine if the compounds discovered in the high-throughput screen described in FIG. 45 attenuate the interaction between the two co-receptors, WNT/LRP6 receptor and PTH1R, at the level of the receptors at or near the cell membrane.

### DETAILED DESCRIPTION

Intermittent administration of parathyroid hormone (PTH) stimulates bone formation, but the precise mechanisms responsible for PTH responses in osteoblasts are incompletely understood. As described herein, the binding of PTH to its receptor, parathyroid hormone 1 receptor (PTH1R), induced association of lipoprotein related protein 6 (LRP6), a co-receptor of Wnt, with PTH1R. The formation of the ternary complex containing PTH, PTH1R, and LRP6 promoted rapid phosphorylation of LRP6, which resulted in the recruitment of axin to LRP6, and stabilization of β-catenin. Activation of PKA is essential for PTH-induced β-catenin stabilization, but not for Wnt signaling. In vivo studies showed that PTH treatment led to phosphorylation of LRP6 and an increase in amount of β-catenin in osteoblasts with a concurrent increase in bone formation in rat. Thus, LRP6 coreceptor is a key element of the PTH signaling that regulates osteoblast activity.

Provided herein are methods of screening for an agent that is a PTH agonist. For example, provided is a method of screening for an agent that is a PTH agonist, the method comprising contacting a cell with LRP6 and the agent to be screened, wherein the cell comprises a PTH1R, and determining the level of LRP6 binding to the PTH1R. An increased level of LRP6 binding to the PTH1R compared to a control indicates the agent is a PTH agonist. Optionally, LRP6 is a modified LRP6 comprising a deletion mutation, including, for example, a truncation mutation. Optionally the modified LRP6 comprises the extracellular and transmembrane domains or portions thereof of wild type LRP6. Optionally, the modified LRP6 does not comprise the intracellular domain(s) of LRP6.

As used herein, a control can be an untreated sample or a sample in the absence of treatment with the agent. A control can include a known value or can be a sample run in parallel with the experimental sample.

Also provided herein are methods of screening for an agent that is a PTH antagonist. For example, the methods comprise contacting a cell with LRP6 and the agent to be screened, wherein the cell comprises a PTH1R, and determining the level of LRP6 binding to the PTH1R. A decreased level of LRP6 binding to the PTH1R compared to a control indicates the agent is a PTH antagonist. Optionally, LRP6 is a modified LRP6 comprising a deletion mutation, including, for example, a truncation. Optionally the modified LRP6 comprises the extracellular and transmembrane domains of wild type LRP6. Optionally, the modified LRP6 does not include the intracellular domain(s) of LRP6.

The cell contacted with LRP6 can, for example, be a primary cell or a cell from an immortalized, transformed cell line. A primary cell can, for example, be cultured from a subject and can include, but is not limited to, a cell selected from the group consisting of epithelial cells, keratinocytes, fibroblasts, hepatocytes, osteoblasts, myocytes, kidney cells, lung cells, thyroid cells, and pancreatic cells. An immortalized, transformed cell line can include, but are not limited to, a cell line selected from the group consisting of HeLa cells, HEK293 cells, Jurkat cells, HepG2 cells, UMR-106 cells, HCT 116 cells, PANC-1 cells, IMR-32 cells, and LNCaP cells.

Optionally, LRP6 and PTH1R are human. Optionally, LRP6 and PTH1R are non-human (e.g., primate, rodent, canine, or feline). There are a variety of sequences that are disclosed on GenBank, at www.pubmed.gov and these sequences and others herein are incorporated by reference in their entireties as are individual subsequences or fragments contained therein. As used herein, LRP6 refers to the LRP6 co-receptor that acts synergistically with the Frizzled (Fz)-receptor family members to bind Wnt and activate downstream signaling in the Wnt signaling pathway. For example, the nucleotide and amino acid sequences or the human LRP6 can be found at GenBank Accession Nos. NM_{—}002336 and NP_{—}002327, respectively. As used herein, PTH1R refers to the PTH1R that binds PTH, which leads to the activation of G protein α subunits that leads to the production of 3′,5′-cyclic adenosine-5′-monophosphate (cAMP) and the activation of phospholipase C (PLC). For example, the nucleotide and amino acid sequences for the human PTH1R can be found at GenBank Accession Nos. NM_{—}000316 and NP_{—}000307, respectively. Thus provided are the nucleotide sequences of LRP6 and PTH1R comprising a nucleotide sequence at least about 70%, 75%, 80%, 85%, 90%, 95%, 98%, 99% or more identical to the nucleotide sequence of the aforementioned GenBank Accession Numbers. Also provided are amino acid sequences of LRP6 and PTH1R comprising an amino acid sequence at least about 70%, 75%, 80%, 85%, 90%, 95%, 98%, 99% or more identical to the sequences of the aforementioned GenBank Accession Numbers.

As with all peptides, polypeptides, and proteins, including fragments thereof, it is understood that additional modifications in the amino acid sequence of the LRP6 and PTH1R polypeptides can occur that do not alter the nature or function of the peptides, polypeptides, or proteins. Such modifications include conservative amino acids substitutions and are discussed in greater detail below.

The polypeptides provided herein have a desired function. LRP6 is a co-receptor that binds PTH1R that has been bound by PTH. Upon formation of a ternary complex, LRP6 recruits axin from the cytoplasm to the cell membrane and promotes stabilization of β-catenin. PTH1R is a receptor that binds PTH and regulates expression of β-catenin. PTH1R regulates expression of β-catenin by binding LRP6. The polypeptides are tested for their desired activity using the in vitro assays described herein.

The polypeptides described herein can be further modified and varied so long as the desired function is maintained. It is understood that one way to define any known modifications and derivatives or those that might arise, of the disclosed genes and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to LRP6 and PTH1R and variants provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol. Biol. 48: 443 (1970), by the search for similarity method of Pearson and Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989), Jaeger et al., Proc. Natl. Acad. Sci. USA 86:7706-7710 (1989), Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations (e.g., due to genetic polymorphism) or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Post-translational modifications can include variations in the type or amount of carbohydrate moieties of the protein core or any fragment or derivative thereof. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from two to six residues are deleted at any one site within the protein molecule. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at lease one residue has been removed and a different residues inserted in its place. Conservative substitutions generally are made in accordance with the following Table 1.

TABLE 1
Amino Acid Substitutions

| | Substitutions |
|---|---|
| Amino Acid | (others are known in the art) |
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Modifications, including the specific amino acid substitutions, are made by known methods. By way of example, modifications are made by site specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

Nucleic acids that encode the polypeptide sequences, variants, and fragments thereof are disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequences.

Isolated nucleic acid molecules can be produced by standard techniques. For example, polymerase chain reaction (PCR) techniques can be used to obtain an isolated nucleic acid containing a nucleotide sequence described herein. Various PCR methods are described, for example, in PCR Primer: A Laboratory Manual, Dieffenbach and Dveksler, eds., Cold Spring Harbor Laboratory Press, 1995. Various PCR strategies also are available by which the site-specific nucleotide sequence modifications described herein can be introduced into a template nucleic acid. Optionally, isolated nucleic acids are chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids disclosed herein also can be obtained by mutagenesis of, e.g., a naturally occurring DNA.

Nucleic acids that encode the polypeptide sequences, variants, and fragments thereof can be cloned into a vector for delivery into the cell. There are a number of compositions and methods which can be used to deliver nucleic acids to cells, either in vitro or in vivo via, for example, expression vectors. These methods and compositions can largely be broken down into two classes: viral based delivery systems and non-viral based delivery systems. Such methods are well known and readily adaptable for use with the compositions and methods described herein.

As used herein, plasmid or viral vectors transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, Sindbis and other RNA viruses, including these viruses with the HIV backbone. Also preferred are any viral families which share the properties of these viruses which make them suitable for use as vectors. Retroviral vectors, in general, are described by Verma, I. M., Retroviral vectors for gene transfer. In Microbiology-1985, American Society for Microbiology, Washington, pp. 229-232 (1985), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma and a number of other tissue sites. Other useful systems include, for example, replicating and host-restricted non-replicating vaccinia virus vectors.

Also provided are expression vectors comprising the disclosed nucleic acids, wherein the nucleic acids are operably linked to an expression control sequence. Suitable vector backbones include, for example, those routinely used in the art such as plasmids, viruses, artificial chromosomes, BACs, YACs, or PACs. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus and most preferably cytomegalovirus, or from heterologous mammalian promoters, e.g. beta actin promoter or EF1 promoter, or from hybrid or chimeric promoters (e.g., cytomegalovirus promoter fused to the beta actin promoter). The early and late promoters of the SV40 virus are conveniently obtained as an SV40 restriction fragment which also contains the SV40 viral origin of replication. The immediate early promoter of the human cytomegalovirus is conveniently obtained as a HindIII E restriction fragment. Of course, promoters from the host cell or related species also are useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs in length, and they function in cis. Enhancers usually function to increase transcription from nearby promoters. Enhancers can also contain response elements that mediate the regulation of transcription. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

Optionally, the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter. Other preferred promoters are SV40 promoters, cytomegalovirus (plus a linked intron sequence), beta-actin, elongation factor-1 (EF-1) and retroviral vector LTR. Optionally the promoter and/or enhancer region can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light.

The vectors also can include, for example, origins of replication, scaffold attachment regions (SARs), and/or markers. A marker gene can confer a selectable phenotype, e.g., antibiotic resistance, on a cell. This marker product is used to determine if the gene has been delivered to the cell and once delivered is being expressed. Examples of marker genes include the E. coli lacZ gene, which encodes B galactosidase, green fluorescent protein (GFP), and luciferase. Examples of suitable selectable markers for mammalian cells are dihydrofolate reductase (DHFR), thymidine kinase, neomycin, neomycin analog G418, hygromycin, blasticidin, and puromycin. When such selectable markers are successfully transferred into a mammalian host cell, the transformed mammalian host cell can survive if placed under selective pressure.

Optionally, LRP6 and/or PTH1R is/are linked to an expression tag. An expression vector can include a tag sequence designed to facilitate manipulation or detection (e.g., purification or localization) of the expressed polypeptide. Tag sequences, such as glutathione S-transferase (GST), polyhistidine (His), myc, hemagglutinin (HA), V5, IgG, T7, or FLAG™ tag (Kodak, New Haven, Conn.) sequences typically are expressed as a fusion with the encoded polypeptide. For example, LRP6 can be linked to the IgG tag, and PTH1R can be linked to the HA tag. Such tags can be inserted anywhere within the polypeptide, including at either the carboxyl or amino terminus. Optionally the expression tag can be a fluorescent protein tag. Fluorescent proteins can, for example, include such proteins as green fluorescent protein (GFP), red fluorescent protein (RFP), cyan fluorescent protein (CFP), yellow fluorescent protein (YFP), and blue fluorescent protein (BFP). Optionally LRP6 can be linked to GFP, and PTH1R can be linked to RFP. Fluorescent proteins can be inserted anywhere within the polypeptide, but are most preferably inserted at either the carboxyl or amino terminus.

The level of LRP6 binding to PTH1R is determined using an assay selected from the group consisting of co-immunoprecipitation assay, immunofluorescent colocalization assay, photobleaching-based fluorescence resonance energy transfer (FRET), and affinity chromatography. Preferably, the level of LRP6 binding to the PTH1R is determined using the immunofluorescent colocalization assay. The analytical techniques used to determine the level of LRP6 binding to the PTH1R are known. See, e.g., Sambrook et al., Molecular Cloning: A Laboratory Manual, 3^{rd} Ed., Cold Spring Harbor Press, Cold Spring Harbor, N.Y. (2001); Wang et al., Annu Rev. Biomed. Eng. 10:1-38 (2008); Kaboord and Perr, Methods Mol. Biol. 424:349-64 (2008); and Fang and Zhang, J. Proteomics 71:284-303 (2008).

Optionally, the methods comprise contacting a cell with a PTH polypeptide or a receptor-binding fragment thereof, a WNT polypeptide, and the agent to be screened, wherein the cell comprises a PTH1R and a nucleotide sequence encoding a reporter protein operably linked to an inducible promoter, wherein the inducible promoter is activated by PTH1R; and determining a level of reporter protein expression. An increased level of reporter protein expression as compared to a control indicates the agent is a PTH agonist. The inducible promoter for example, can comprise at least one T-cell factor/lymphoid enhancer factor (TCF/LEF) binding site.

Optionally, the methods comprise contacting a cell with a PTH polypeptide or a receptor-binding fragment thereof, a WNT polypeptide, and the agent to be screened, wherein the cell comprises a PTH1R; and determining a level of secreted osteosclerostin polypeptide. An increased level of secreted osteosclerostin polypeptide as compared to a control indicates the agent is a PTH agonist.

Optionally, the methods comprise contacting a cell with a PTH polypeptide or a receptor-binding fragment thereof, a WNT polypeptide, and the agent to be screened, wherein the cell comprises a PTH1R and a nucleotide sequence encoding a reporter protein operably linked to an inducible promoter, wherein the inducible promoter is activated by PTH1R; and determining a level of reporter protein expression. A decrease in reporter protein expression as compared to a control indicates the agent is a PTH antagonist. The inducible promoter, for example, can comprise at least one T-cell factor/lymphoid enhancer factor (TCF/LEF) binding site.

Optionally, the methods comprise contacting a cell with a PTH polypeptide or a receptor-binding fragment thereof, a WNT polypeptide, and the agent to be screened, wheiren the cell comprises a PTH1R; and determining a level of secreted osteosclerostin polypeptide. A decreased level of secreted osteosclerostin polypeptide as compared to a control indicates the agent is a PTH antagonist.

The Wnt polypeptide can be a full-length polypeptide or a receptor-binding fragment thereof. Optionally, the Wnt polypeptide or receptor-binding fragment thereof comprises a Wnt3a polypeptide or receptor-binding fragment thereof.

The reporter protein can be selected from the group consisting of green fluorescent protein (GFP), yellow fluorescent protein (YFP), cyan fluorescent protein (CFP), red fluorescent protein (RFP), chloramphenicol acetyl transferase (CAT), and luciferase. Optionally, the reporter protein is luciferase. The level of reporter protein expression is determined using an assay selected from the group consisting of a Western blot, an enzyme-linked immunosorbent assay (ELISA), an AlphaLISA® (Perkin-Elmer; Waltham, Mass.) assay, a radioimmunoassay, an enzyme immuno-assay, and a fluorescent imaging assay.

Optionally, the cell is an osteosarcoma cell. The osteosarcoma cell, for example, can be a UMR-106 cell.

The agent, for example, can be selected from the group consisting of a small molecule, a polypeptide, a nucleic acid molecule, a peptidomimetic, or a combination thereof.

Also provided is a method of treating or preventing a skeletal disorder in a subject, wherein the skeletal disorder is characterized by proliferative bone growth. Skeletal disorders characterized by proliferative bone growth can, for example, include Paget's disease, bone tumors (e.g., osteoma, osteochondroma, aneurismal bone cyst, and fibrous dysplasia), and osteopetrosis. The method comprises identifying a subject with or at risk of developing the skeletal disorder and administering to the subject an agent that inhibits the binding of LRP6 to PTH1R. Optionally, the agent to be administered can be the agent identified in the screen for PTH antagonists as described herein. Optionally, the agent is selected from the group consisting of a small molecule, a polypeptide, a nucleic acid molecule, a peptidomimetic, or a combination thereof. Optionally, the agent can be a polypeptide. The polypeptide can, for example, comprise the extracellular domain of LRP6. The polypeptide can also comprise an antibody. Optionally, the agent can be a nucleic acid molecule. The nucleic acid molecule can, for example, be an LRP6 or PTH inhibitory nucleic acid molecule. The LRP6 or PTH1R inhibitory nucleic acid molecule can comprise a short interfering RNA (siRNA) molecule, a microRNA (miRNA) molecule, or an antisense molecule.

As used herein, a LRP6 or PTH inhibitory nucleic acid sequence can be a siRNA sequence or a miRNA sequence. A 21-25 nucleotide siRNA or miRNA sequence can, for example, be produced from an expression vector by transcription of a short-hairpin RNA (shRNA) sequence, a 60-80 nucleotide precursor sequence, which is processed by the cellular RNAi machinery to produce either an siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion, Inc. (Austin, Tex.). A siRNA sequence preferably binds a unique sequence within the LRP6 or PTH mRNA with exact complementarity and results in the degradation of the LRP6 or PTH1R mRNA molecule. A siRNA sequence can bind anywhere within the mRNA molecule. A miRNA sequence preferably binds a unique sequence within the LRP6 or PTH1R mRNA with exact or less than exact complementarity and results in the translational repression of the LRP6 or PTH1R mRNA molecule. A miRNA sequence can bind anywhere within the mRNA molecule, but preferably binds within the 3′UTR of the mRNA molecule. Methods of delivering siRNA or miRNA molecules are known in the art. See, e.g., Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discov. 8(2)129-38 (2009).

As used herein, a LRP6 or PTH inhibitory nucleic acid sequence can be an antisense nucleic acid sequence. Antisense nucleic acid sequences can, for example, be transcribed from an expression vector to produce an RNA which is complementary to at least a unique portion of the LRP6 or PTH1R mRNA and/or the endogenous gene which encodes LRP6 or PTH1R. Hybridization of an antisense nucleic acid molecule under specific cellular conditions results in inhibition of LRP6 or PTH1R protein expression by inhibiting transcription and/or translation.

Also provided is a method of treating or preventing a skeletal disorder in a subject, wherein the skeletal disorder is characterized by reduced bone density. Skeletal disorders characterized by reduced bone density can, for example, include, but are not limited to, osteoporosis, osteitis fibrosa cystica, and osteochondritis dissecans. The method comprises identifying a subject with or at risk of developing the skeletal disorder and administering to the subject an agent that stimulates the binding of LRP6 to PTH1R. Optionally, the agent to be administered can be the agent identified in the screen for PTH agonists as described herein. Optionally, the agent is a small molecule, a polypeptide, or a combination thereof. Optionally, the agent is a small molecule. Optionally, the agent is a polypeptide. Optionally, the polypeptide is an antibody.

The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

Provided herein are compositions containing the provided agent and a pharmaceutically acceptable carrier. The herein provided compositions are administered in vitro or in vivo. By pharmaceutically acceptable carrier is meant a material that is not biologically or otherwise undesirable, i.e., the material is administered to a subject, e.g., with an agent to treat or prevent a skeletal disorder, without causing any undesirable biological effects or interacting in a deleterious manner with any of the other components of the pharmaceutical composition in which it is contained. The carrier is selected to minimize any degradation of the active ingredient and to minimize any adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained release preparations such as semipermeable matrices of solid hydrophobic polymers containing the immunogenic polypeptides. Matrices are in the form of shaped articles, e.g., films, liposomes or microparticles. Certain carriers may be more preferable depending upon, for instance, the route of administration and concentration of composition being administered. Carriers are those suitable for administration of the agent, e.g., the agent that promotes or the agent that inhibits binding of LRP6 and PTH1R, to humans or other subjects.

The compositions are administered in a number of ways depending on whether local or systemic treatment is desired, and on the area to be treated. The compositions are administered via any of several routes of administration, including, topically, orally, parenterally, intravenously, intraperitoneally, intramuscularly, subcutaneously, intracavity, transdermally, intrahepatically, intracranially, nebulization/inhalation, or by instillation via bronchoscopy.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Optionally, the composition is administered by oral inhalation, nasal inhalation or intranasal mucosal administration. As used herein, these terms mean delivery of the compositions into the nose and nasal passages through one or both of the nares and can comprise delivery by a spraying mechanism or droplet mechanism, or through aerosolization of the nucleic acid or vector.

Formulations for topical administration include ointments, lotions, creams, gels, drops, suppositories, sprays, liquids and powders. Conventional pharmaceutical carriers, aqueous, powder or oily bases, thickeners and the like are optionally necessary or desirable.

Compositions for oral administration include powders or granules, suspensions or solutions in water or non-aqueous media, capsules, sachets, or tablets. Thickeners, flavorings, diluents, emulsifiers, dispersing aids or binders are optionally desirable.

Provided herein are methods of treating a skeletal disorder in a subject. Such methods include administering one or more agents and combinations thereof to the subject. Optionally, the agents are contained within a pharmaceutical composition as described above. Optionally, the agent is a nucleic acid molecule or a polypeptide, which can be administered by a vector comprising the nucleic acid molecule or a nucleic acid sequence encoding the polypeptide. Methods of administration by a vector are described above.

As used herein, the term peptide, polypeptide or protein is used to mean a molecule comprised of two or more amino acids linked by a peptide bond. Protein, peptide, and polypeptide are also used herein interchangeably to refer to amino acid sequences. It should be recognized that the term polypeptide or protein is not used herein to suggest a particular size or number of amino acids comprising the molecule and that a polypeptide of the disclosure can contain up to several amino acid residues or more.

As used throughout, by a subject is meant an individual. Thus, the subject can include, for example, domesticated animals, such as cats and dogs, livestock (e.g., cattle, horses, pigs, sheep, and goats), laboratory animals (e.g., mice, rabbits, rats, and guinea pigs), mammals, non-human mammals, primates, non-human primates, rodents, birds, reptiles, amphibians, fish, and any other animal. The subject can be a mammal such as a primate or a human. The term subject also includes individuals of different ages. Thus, a subject includes an infant, child, teenager or adult.

A subject at risk of developing a skeletal disease or disorder can be genetically predisposed to the disease or disorder, e.g., have a mutation in a gent that causes the disease or disorder or have a family history of the disease or disorder. Additionally, the subject may have one or more risk factors for developing the skeletal disease or disorder. Risk factors for osteoporosis include the following factors, age (more common in older subject), sex (more common in female subjects), family history, stature (low body weight or small frame), ethnicity (Caucasian, Asian, Latin), history of fractures, menopause, low estrogen and/or testosterone levels, amenorrhea, diet (e.g., low calcium intake, high protein intake, high salt intake), inactivity, smoking, alcohol consumption, certain medications (steroids), history of anorexia, celiac disease, hypothyroidism, hyperthyroidism, and inflammatory bowel disease. A subject at risk of developing a skeletal disease or disorder may have symptoms or signs of early onset for the disease or disorder. A subject with a skeletal disease or disorder has one or more symptoms of the disease or disorder and has been diagnosed with the disease or disorder.

The methods and agents as described herein are useful for both prophylactic and therapeutic treatment. For prophylactic use, a therapeutically effective amount of the agents described herein are administered to a subject prior to onset (e.g. before obvious signs of the skeletal disorder) or during early onset (e.g. upon initial signs and symptoms of the skeletal disorder). Prophylactic administration can occur for several days to years prior to full manifestation of symptoms of the skeletal disorder. Prophylactic administration can be used, for example, in the preventative treatment of subjects diagnosed with a genetic predisposition to the skeletal disorder. Therapeutic treatment involves administering to a subject a therapeutically effective amount of the agents described herein after diagnosis of the skeletal disorder.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered by one or more dose administrations daily, for hours or several days. Guidance can be found in the literature for appropriate dosages for given classes of pharmaceutical agents.

As used herein the terms treatment, treat or treating refer to a method of reducing the effects of a disease or condition or symptom of the disease or condition. Thus in the disclosed method, treatment can refer to a 10%, 20%, 30%, 40%, 50%, 60%, 70%, 80%, 90% or 100% reduction in the severity of an established disease or condition or symptom of the disease or condition. For example, a method for treating a disease is considered to be a treatment if there is a 10% reduction in one or more symptoms of the disease in a subject as compared to control. Thus the reduction can be a 10%, 20%, 30%, 40%, 50%, 60%, 70%, 80%, 90%, 100% or any percent reduction in between 10% and 100% as compared to native or control levels. It is understood that treatment does not necessarily refer to a cure or complete ablation of the disease, condition or symptoms of the disease or condition.

As used herein, the terms prevent, preventing and prevention of a disease or disorder refer to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a substitution or modification is disclosed and discussed and a number of other substitutions or modifications that can be made, each and every combination and permutation can be combined, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

The examples below are intended to further illustrate certain aspects of the methods and compounds described herein, and are not intended to limit the scope of the claims.

### EXAMPLES

### General Methods

cDNA Constructs

PTH1R tagged with HA was subcloned into pcDNA3.1. cDNAs from human LRP5 (Mao et al., Mol. Cell. 7:801-9 (2001)) and LRP6 (Tamai et al., Nature 407:530-35 (2000)) tagged with HA and VSVG were subcloned into pCMV and pCS2⁺, respectively. LRP6N+T (LRP6 N-terminal plus the transmembrane domain) and LRP6T+C (LRP6 transmembrane domain plus C-terminal) tagged with VSVG were subcloned into pCS2⁺. LRP6N+1479m, LRP6N+1490m, LRP6N+1493m and LRP6N+1496m were generated by mutagenesis of either the serine (at amino acid 1490 or amino acid 1496) or threonine (at amino acid 1479 or amino acid 1493) to alanine LRP6N-IgG was generated by fusing the LRP6 extracellular domain with IgG (Tamai et al., Nature 407:530-35 (2000)). si-GFP (Wan et al., Am. J. Pathol. 166:1379-92 (2005)) and si-LRP6 plasmids were generated using a BS/U6 vector. Briefly, a 22-nucleotide oligo (oligo 1) corresponding to nucleotides 2981 to 3002 of the human LRP6 coding region was first inserted into the BS/U6 vector digested with ApaI (blunted) and HindIII. The inverted motif that contains the six-nucleotide spacer and five Ts (oligo 2) was then subcloned into the HindIII and EcoRI sites of the intermediate plasmid to generate BS/U6/LRP5/6.

### Primary Osteoblast Isolation and Culture

Osteoblasts were isolated by digestion of calvaria of newborn mice as described (Wang et al., J. Clin. Invest. 117:1616-26 (2007)). Briefly, calvaria were incubated with 10 ml of digestion solution containing 1.8 mg/ml of collagenase type I (Worthington Biochemical Corp.; Lakewood, N.J.) for 15 minutes at 37° C. under constant agitation. The supernatant was then harvested, replaced with fresh collagenase and the digestion repeated an additional 4 times. Digestion solutions containing the osteoblasts were pooled together. After centrifugation, osteoblasts were obtained and cultured in α-Minimal Essential Media (MEM) containing 10% FBS and 1% penicillin/streptomycin at 37° C. in a humidified incubator supplied with 5% CO₂.

### Cell Culture, Conditioned Media, Transfection and Luciferase Reporter Assays

HEK293, UMR-106 and mouse embryonic fibroblast (MEF) cells were maintained in Dulbecco's Modified Eagle Medium (DMEM) with 10% Fetal Calf Serum (FCS). Mouse Wnt3a conditioned medium (Wnt3a CM) was produced from mouse L cells stably transfected with mouse Wnt3a (American Type Culture Collection; Manassas, Va.) and control conditioned medium (Control CM) was from non-transfected L cells. IgG, LRP6N-IgG, DKK1, Sclerostin, VSVG-LRP6N and Myc-Fz8CRD conditioned media were produced from HEK 293 cells transfected with the individual plasmids. Transfections were carried out using lipofectamine reagent (Invitrogen; Carlsbad, Calif.). Luciferase assays were carried out in either UMR-106 or HEK 293 cells as described previously (Wan et al., Am. J. Pathol. 166:1379-92 (2005)), with 0.3 μg of TCF-Luc reporter plasmid plus 50 ng of Renilla luciferase plasmid (internal control) per well in the 12-well plate. Experiments were repeated at least three times with triplicate for each experiment.

### High-Throughput Screen

Osteosarcoma cells were plated in a 96 well plate. The cells were transiently transfected with the TCF/LEF luciferase reporter using LipofectAMINE PLUS (Invitrogen; Carlsbad, Calif.). The cells were incubated overnight at 37° C. in OptiMEM-1 serum-free medium with the transfection reagents. The cells were washed and serum-containing medium was added to the cells for 8 hours. The serum-containing medium was replaced with serum-free medium and the cells were stimulated overnight with PTH and WNT3a. Supernatant from the stimulated cells was collected for detection of osteosclerostin, and the cells were subjected to a luciferase assay as previously described (Wan et al., Am. J. Pathol. 166:1379-92 (2005)).

### Ostesclerostin Detection Assay

Following treatment of the cells, cell assay media is collected and transferred to 96-well (half-area well) microtiter plates to perform an AlphaLISA® assay (Perkin-Elmer; Waltham, Mass.). AlphaLISA® acceptor beads coated with an anti-osteosclerostin antibody are loaded and allowed to bind to osteosclerostin. Streptavidin-coated donor beads, preloaded with a biotinylated anti-osteosclerostin antibody that recognizes a different domain of osteosclerostin are added. Following additional incubation to allow donor bead binding to osteosclerostin, the sample is excited with light at 620 nm wavelength. Photosensitizers present in the donor beads generate free oxygen that travels to the acceptor beads, where the oxygen reacts with a derivative to produce a chemiluminescent signal. The free oxygen generated by donor beads can only travel a very short distance in solution, ensuring that chemiluminescence is only produced when donor and acceptor beads are brought into close proximity through osteosclerostin binding.

### Cell Fractionation, Coimmunoprecipitation and Western Blot Analysis

Cells were harvested in cavitation buffer (5 mM HEPES, pH 7.4, 3 mM MgCl₂, 1 mM EGTA, 250 mM sucrose) containing protease and phosphatase inhibitors and homogenized by nitrogen cavitation (200 p.s.i., for 5 minutes) in a cell disruption bomb (Parr Instrument Co.; Moline, Ill.). The cell homogenate was centrifuged twice at 700 g for 10 minutes to pellet the nuclei. The supernatant was further centrifuged at 100,000 g (Beckman SW50.1 rotor) for 1 hour to separate the membrane and cytosol fractions, and the resulting membrane pellet was washed three times with cavitation buffer before use in the assays (Zhang et al., Biochem. J. 343:541-9 (1999)). Immunoprecipitation and Western blot analysis of cell lysates were performed as described previously (Wan et al., Am. J. Pathol. 166:1379-92 (2005)).

### Cell Surface Binding by Immunofluorescence Colocalization Assay

Cells were transfected with HA-PTH1R and treated with IgG conditioned medium or LRP6N-IgG conditioned medium for 1 hour followed by PTH (1-84) treatment for 15 minutes. Cells were then washed with phosphate buffered saline (PBS), fixed with 4% paraformaldehyde, permeabilized with 0.1% Triton X-100, and incubated with primary antibody followed by incubation with chromophore-conjugated secondary antibody. Digital pictures were taken using an Olympus, IX TRINOC camera fitted to an Olympus, IX70 Inverted Research Microscope (Olympus; Center Valley, Pa.) with objective lenses of Hoffman Modulation Contrast®, HMC 10 LWD PL FL, 0.3NA ∝/1, at room temperature, and processed using MagnaFire® SP imaging software. A Zeiss TCs SP2 system was used for confocal imaging. The ratios of the number of cells showing in green to the number of cells showing in red were calculated. For each treatment, 100 cells on each of three different slides were analyzed.

### Metabolic ³²P Labeling and In Vivo Phosphorylation Assays

Cells were transfected with expression plasmids and were washed twice with phosphate-free DMEM containing 2% dialyzed FCS, incubated in the same medium for 4 hours, and then labeled with 1 mCi/ml [³²P]orthophosphate (PerkinElmer; Waltham, Mass.) for an additional 2 hours. The ³²P-labeled cells were then washed with ice-cold PBS and lysed with radioimmunoprecipitation assay buffer. VSVG-LRP6 was immunoprecipitated with anti-VSVG, and the resultant precipitates were separated by 8.5% SDS-PAGE. Gels were dried and exposed to Biomax Mᵣ or MS film (Eastman Kodak Co.; Rochester, N.Y.). After autoradiographic analysis, dried gels were rehydrated with transfer buffer, and transferred onto PVDF membranes. For equal loading confirmation, the transfected VSVG-LRP6 was visualized by the ECL^{Plus} Western blotting detection system (Amersham Biosciences; Pittsburgh, Pa.).

### Animals

For the experiments in which rats or mice were administered PTH as single-dose injection, five-month-old male Sprague Dawley rats (Charles River Laboratories; Wilmington, Mass.) or two-month-old male C57BL/J6 mice (Jackson Laboratory; Bar Harbor, Me.) (6 per group) were administered a single dose of either vehicle (1 mM acetic acid in sterile PBS) or PTH (1-34) (Bachem Inc.; Torrance, Calif.) at 40 μg/kg in a volume of 100 μl. In the mouse model, mouse recombinant Wnt3a (R&D Systems; Minneapolis, Minn.) was injected at 25 μg/kg in a volume of 100 μl. All treatments were through bolus intravenous injection via the tail vein. Rats/mice were sacrificed at 0.5, 2, 8 and 24 hours after injection.

In the intermittent injection model, PTH (1-34) (40 μg/kg per day) or vehicle (equivalent volume of 1 mM acetic acid in sterile PBS) in a final volume of 100 μl was given daily by subcutaneous injection for 6 weeks to two-month-old male C57BL/J6 mice (6 per group). In the continuous infusion model, ALZET® Osmotic Pumps (Model 2004, DURECT Corp., Cupertino, Calif.) were implanted subcutaneously into the backs of mice under anesthesia. Continuous infusion of PTH (1-34) or vehicle (equivalent volume of 1 mM acetic acid in sterile PBS) was conducted to release 40 μg/kg per day at the rate of 0.25 μl/h for 6 weeks. To ensure continuous administration of active PTH (1-34), the original pump was removed and replaced by a new one in a different subcutaneous site every 2 weeks.

### Immunohistochemical Analysis of the Bone Tissue

Formalin-fixed femur or tibia tissue sections of 5 μm thickness from rats or mice were processed with antigen retrieval and hydrogen peroxide treatment prior to incubation with primary monoclonal antibody specific for β-catenin (BD Biosciences; San Jose, Calif.), goat polyclonal antibody sclerostin (R&D Systems; Minneapolis, Minn.) or rabbit polyclonal phosphorylated LRP6 (Ab1490) for 1 hour at room temperature or overnight at 4° C. Negative controls were obtained by replacing the primary antibodies with irrelevant control isotype IgG. Antibody detection was accomplished using the biotin-streptavidin horseradish peroxidase (for β-catenin and sclerostin) or alkaline phosphatase (for Ab1490) (EnVision™ System; Dako, Denmark). β-catenin and sclerostin staining was based on peroxidase (HRP) using DAB as chromogen. Phospho-LRP6 staining was based on alkaline phosphotase (AP) using Permanent Red as chromogen. The sections were then counterstained with hematoxylin. Isotype-matched negative control antibodies (R&D Systems; Minneapolis, Minn.) were used under the same conditions. Osteoblasts/preosteoblasts were observed at the bone surface with large, spherical and basal mononucleus. Only those specimens in which greater than 10% of the cells were stained were considered as positive. In the rat model, numbers of total osteoblasts and numbers of β-catenin- or p-LRP6-positive osteoblasts were counted in three random high power fields at metaphysis subjacent to the epiphyseal growth plates or the diaphyseal hematopoietic bone marrow per specimen, and a total of six specimens in each group were used. In the mouse model, numbers of total osteoblasts and numbers of β-catenin-positive osteoblasts were counted in three random high power fields in a 2-mm square, 1 mm distal to the lowest point of the growth plate in the secondary spongiosa. Numbers of total osteocytes and numbers of sclerostin-positive osteocytes were counted in three random high power fields per trabecular bone section or cortical bone section, and a total of six specimens in each group were used.

### Quantitative Real-Time Polymerase Chain Reaction (PCR)

Cells were homogenized using Trizol reagent (Invitrogen; Carlsbad, Calif.), and total RNA was extracted according to the manufacturer's protocol. cDNA was produced and quantitative real-time PCR was performed in an iCycler real-time PCR machine using iQ SYBR Green supermix (Bio-Rad;). Primers are as follows: GAPDH forward: 5′-GGGTGTGAACCACGAGAAAT-3′ (SEQ ID NO:1), GAPDH reverse: 5′-CCTTCCACAATGCCAAAGTT-3′ (SEQ ID NO:2), Osteocalcin forward: 5′-CTTGGTGCACACCTAGCAGA-3′ (SEQ ID NO:3), Osteocalcin reverse: 5′-CTCCCTCATGTGTTGTCCCT-3′ (SEQ ID NO:4), RANKL forward: 5′-CCAAGATCTCTAACATGACG-3′ (SEQ ID NO:5), and RANKL reverse: 5′-CACCATCAGCTGAAGATAGT-3′ (SEQ ID NO:6). The quantity of RANKL and Osteocalcin mRNA in each sample was normalized using the Cᵣ (threshold cycle) value obtained for the GAPDH mRNA amplifications.

### Fluorescence Resonance Energy Transfer (FRET) Procedure

PTH1R and BMPRII cDNAs were cloned into ECFP-N1, and LRP6 and mLRP4T100 cDNAs were cloned into EYFP—N1 (Clonetech; Palo Alto, Calif.) expression vectors. These vectors were modified by site-directed mutagenesis that prevents the self-dimerization (Bhatia et al., Proc. Natl. Acad. Sci. USA 102:15569-74 (2005)). CFP and YFP were fused at the C-termini of the receptors. Because CFP-PTH1R or CFP-BMPRII (the fluorescent FRET donors) is quenched when in the proximity of YFP-LRP6 or YFP-mLRP4T100 (the acceptors), FRET efficiency can be measured by comparing donor fluorescence pre- and post-photobleaching of the acceptor. An increase in donor fluorescence after acceptor photobleaching indicates that donor and acceptor fluorophores were within FRET range. HEK293 cells on coverslips in 35 mm dishes were cotransfected with 0.1 μg of each plasmid. Cells were observed using Leica TCS SP II AOBS laser-scanning confocal microscope. An excitation wavelength of 405 nm and an emission range of 416-492 nm, and an excitation wavelength of 514 nm and an emission range of 525-600 nm were used to acquire images of CFP and YFP, respectively. YFP was photobleached by using full power of the 514 nm line for 1-2 minutes. An image of CFP and YFP fluorescence after photobleaching was obtained by using the respective filter sets. Images were representatives of three experiments. The FRET efficiencies were calculated according to: FRET eff %=[(Donorₚₒₛₜ−Donorₚᵣₑ)/(Donorₚₒₛₜ)]

### Xenopus Embryo Manipulation

RNAs for microinjection were synthesized using SP6 mMessage mMachine in vitro transcription kit (Ambion; Austin, Tex.). RNAs were injected into the marginal zone region of two ventral blastomeres of four-cell stage embryos, and the phenotype of the embryos was observed at the tadpole stages. The doses of RNAs used were: 200 pg LRP6, 2 pg PTH and 50 pg PTH1R.

### In Vitro Kinase Assays

GST, GST-LRP5C and GST-LRP6C were purified from bacterial lysates by absorption to glutathione-agarose beads. GST, GST-LRP5C and GST-LRP6C beads were washed with phosphorylation buffer (25 mM Tris-HCl, pH 7.5, 10 mM MgCl₂, 2 mM MnCl₂, 0.4 mM EDTA, 1 mM dithiothreitol, 2 mM orthovanadate, 10 mM NaF, 5 mM β-glycerophosphate, and 10 μM ATP) containing a protease inhibitor mixture (1 mM phenylmethylsulfonyl fluoride and 10 μg/ml antipain, chymostatin, leupeptin, and pepstatin A). [γ32P]ATP was then added to the mixture and incubated for 30 minutes at 30° C. with PKA catalytic subunit. Phosphorylation status was analyzed on an 8.5% SDS-PAGE gel and autoradiography.

### Alkaline Phosphatase Activity Assays

Cells were cultured in osteogenic induction medium (100 nM ascorbic acid, 10 mM glycerophosphate and 100 ng/ml BMP2) for 5 days. ALP activity was determined using the ALP activity assay kit (APF-1KT, Sigma) according to the manufacturer's protocol.

### Statistical Analysis

Data were analyzed using Student's t test and are expressed as the mean±SEM.

### Example 1

### PTH Induces β-Catenin Stabilization in Osteoblasts

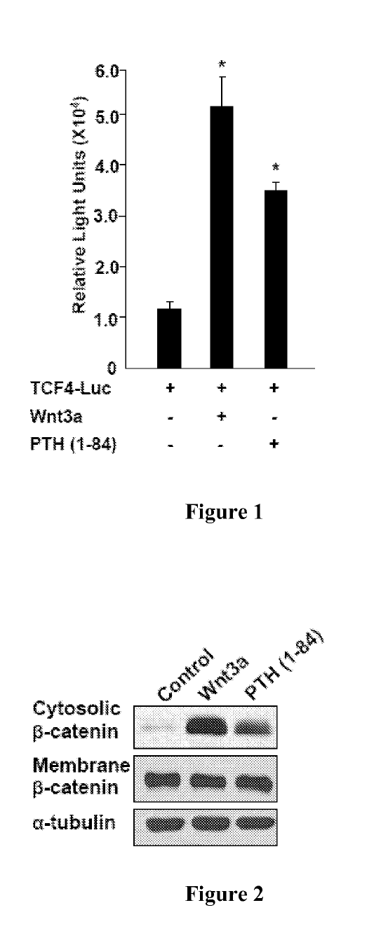

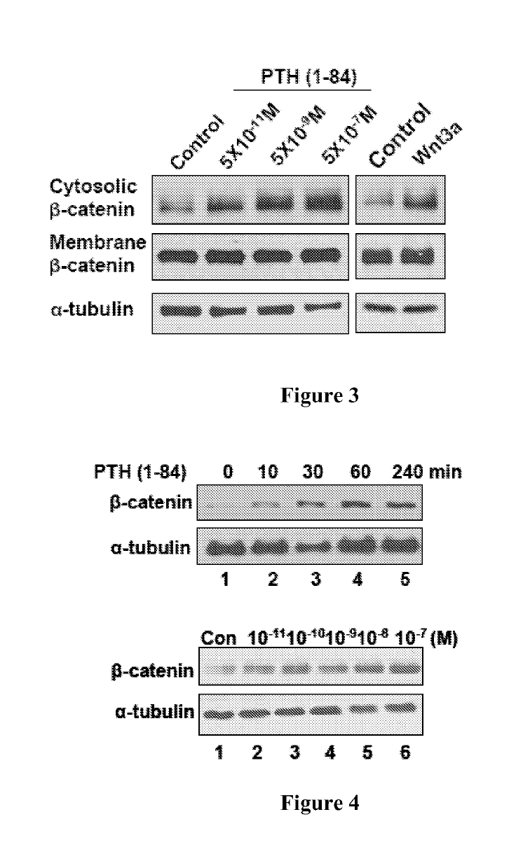

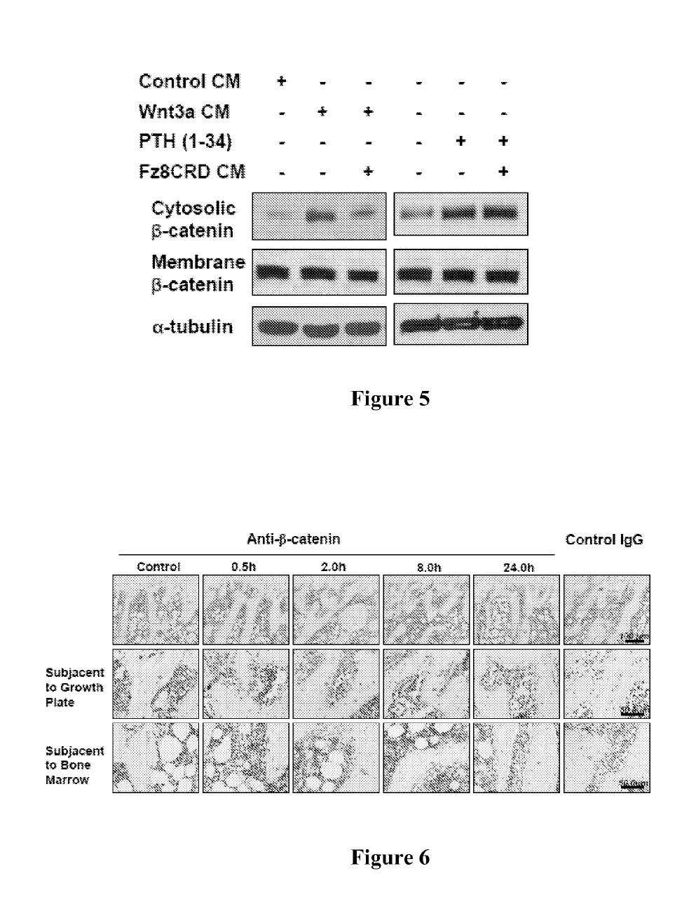

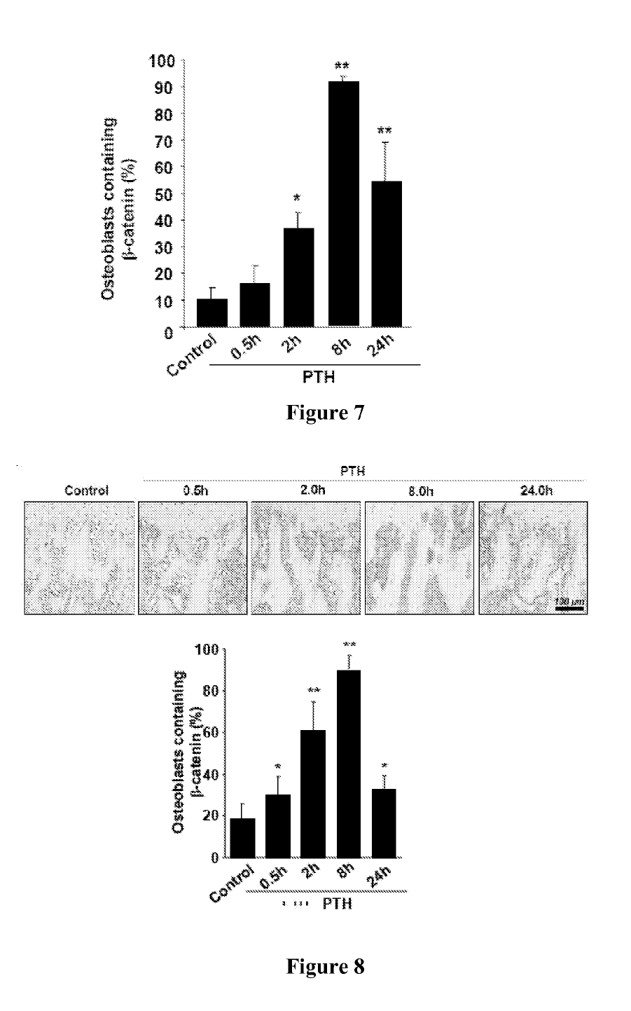

To determine whether PTH regulates expression of β-catenin, the effects of PTH on β-catenin levels in rat UMR-106 osteoblastic cells were examined. It was found that PTH stimulated the transcription of a luciferase reporter bearing TCF/LEF binding elements (FIG. 1), and enhanced the abundance of β-catenin in the cytosol (FIG. 2), whereas the unrelated peptide had no such effects. Similarly, PTH enhanced the levels of β-catenin in the cytosol in a concentration- and time-dependent manner in both mouse calvarial primary preosteoblasts (FIG. 3) and HEK 293 cells (FIG. 4). β-catenin accumulation in the cytosol induced by PTH is so rapid that the effect is unlikely to be mediated through synthesis of Wnt ligands or sensitization of Wnt-stimulated signaling. Indeed, Fz8CRD, a competitive inhibitor of the Wnt receptor Fz (Hsieh et al., Proc. Natl. Acad. Sci. USA 96:3546-51 (1999)) inhibited Wnt3a-, but not PTH-elevated β-catenin level (FIG. 5), thus excluding the possibility of the involvement of Wnts. To test whether PTH stimulates β-catenin in vivo, the effects of PTH (1-34) administered as a single dose to five month-old rats was analyzed. PTH (1-34) is a C-terminal-truncated synthetic analog of PTH with an anabolic effect on bone formation in humans (Treager et al., Endocrinology 93:1349-53 (1973); and Potts et al., Am. J. Med. 50:639-49 (1971)). Immunohistochemistry analysis of sections of the trabecular bone indicated that PTH induced expression of β-catenin in preosteoblasts and osteoblasts on the bone surface within hours (FIG. 6 and FIG. 7). At 8 hours after injection, positive staining of β-catenin was observed in most osteoblasts (99.08±0.57%) at the metaphysis subjacent to the epiphyseal growth plates and about 90.24±0.68% of the osteoblasts at the diaphyseal bone marrow. Similar experiments were carried out using two month-old male mice and similar temporal β-catenin expression patterns were obtained in the mice injected with PTH (FIG. 8).

### Example 2

### LRP6 Forms a Complex with PTH/PTH1R

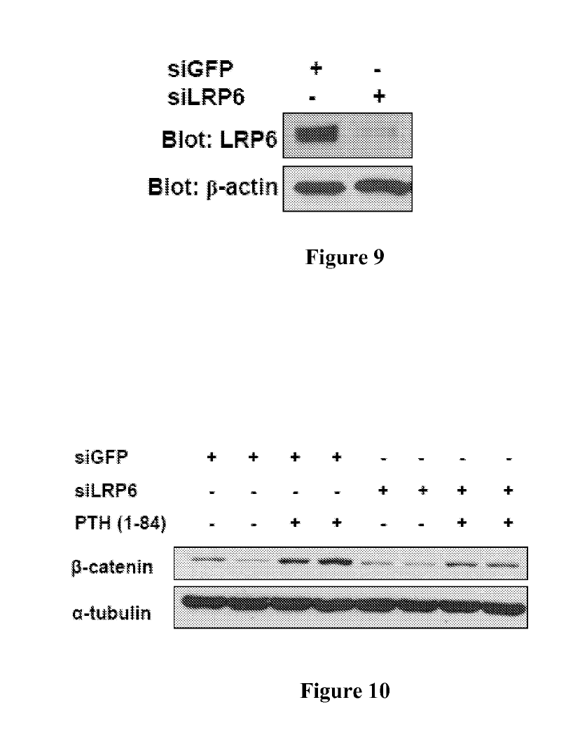

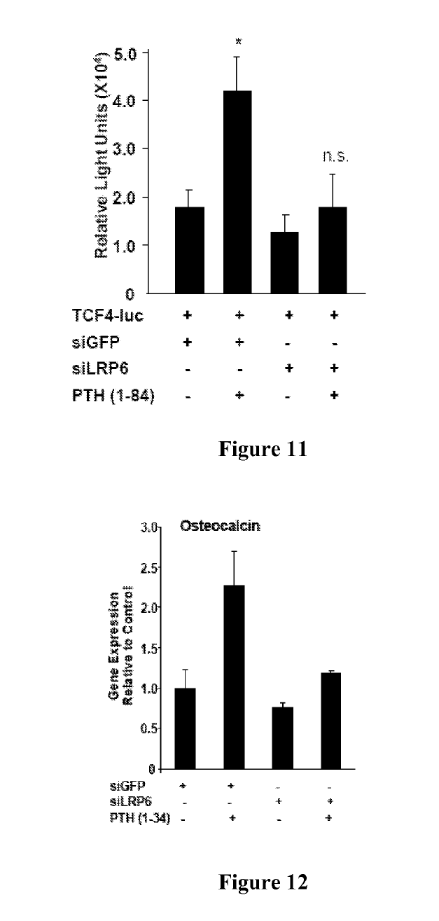

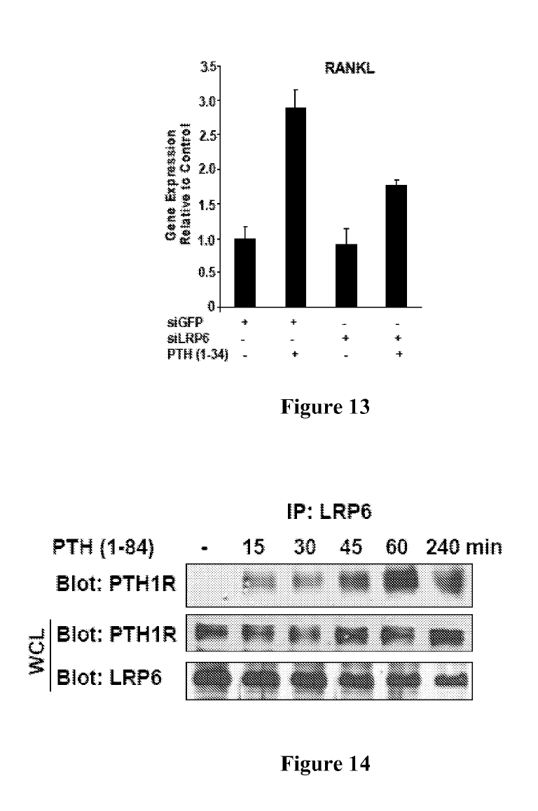

The rapid enhancement of β-catenin protein levels in response to PTH treatment both in vitro and in vivo suggest that PTH may have a direct effect on the signaling components that promote the stabilization of β-catenin. Both LRP5 and LRP6 are key components in activating β-catenin signaling in canonical Wnt pathway. Recent studies reported that PTH anabolic effect was not affected in LRP5 KO mice (Sawakami et al., J. Biol. Chem. 281:23698-711 (2006); and Iwaniec et al., J. Bone Miner. Res. 22:394-402 (2007)), indicating that LRP5 is not essential for the stimulatory effects of PTH on bone formation. To study whether inactivation of LRP6 would affect PTH-elevated β-catenin level, siRNA complementary to lrp6 mRNA was introduced to the cells. Reduction of LRP6 (FIG. 9) attenuated PTH-stimulated accumulation of β-catenin in the cytosol (FIG. 10) and TCF/LEF luciferase activity (FIG. 11). PTH-stimulated mRNA expressions of osteocalcin and RANKL, downstream target genes of PTH that are pertinent to osteoblast differentiation, were also inhibited by siRNA (FIGS. 12 and 13). The results indicate that LRP6 is a critical mediator for PTH-induced β-catenin stabilization.

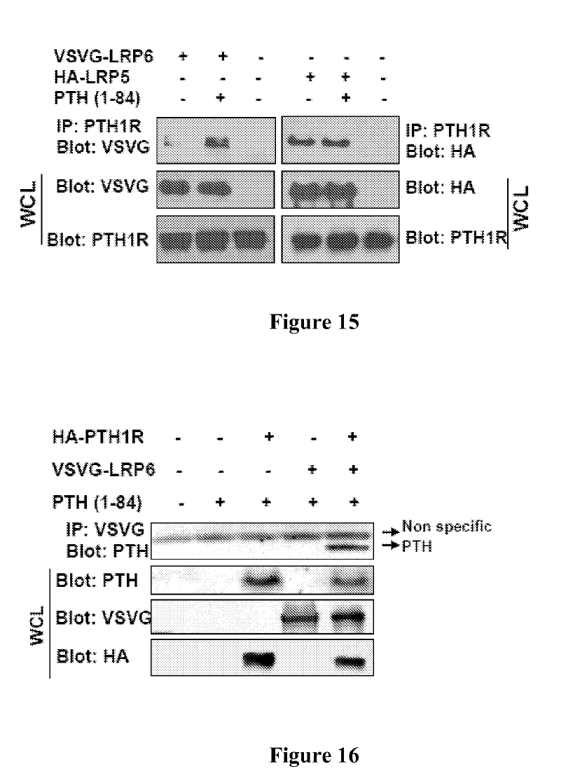

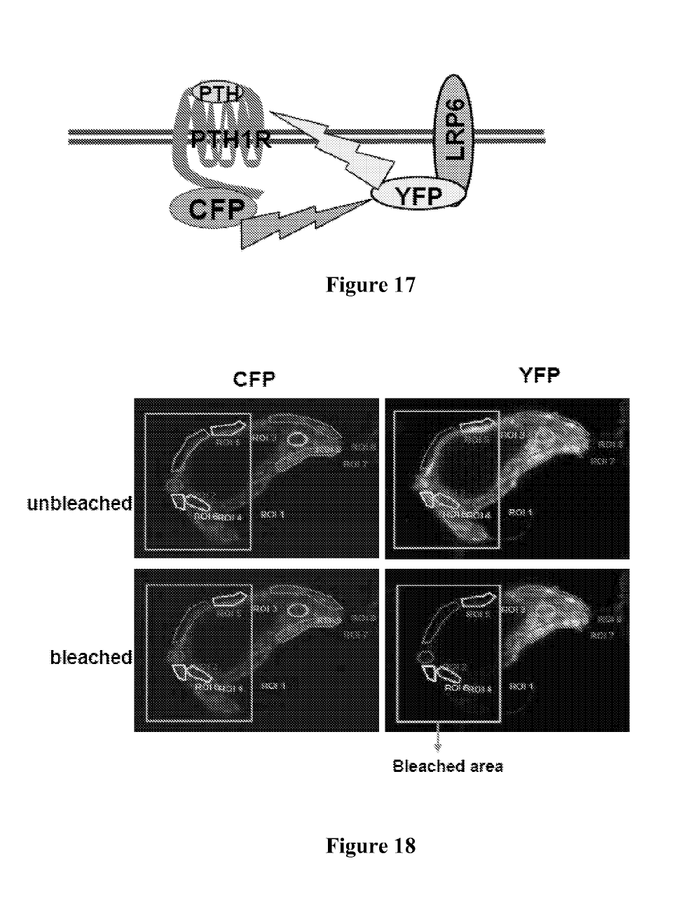

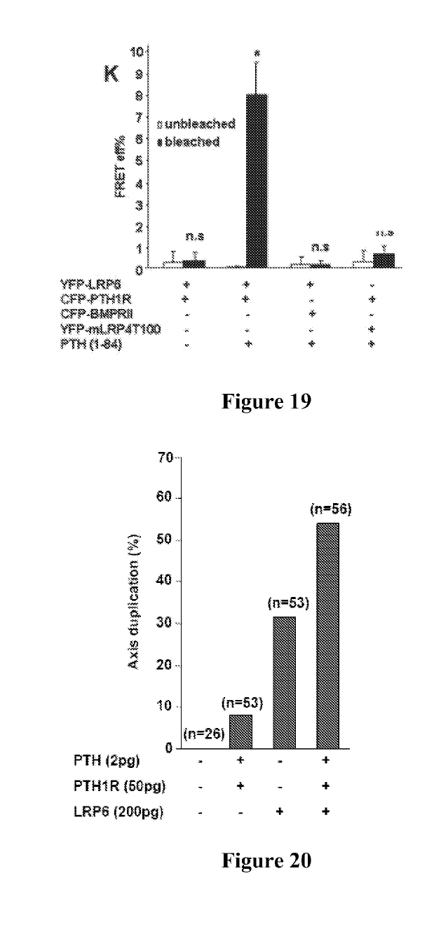

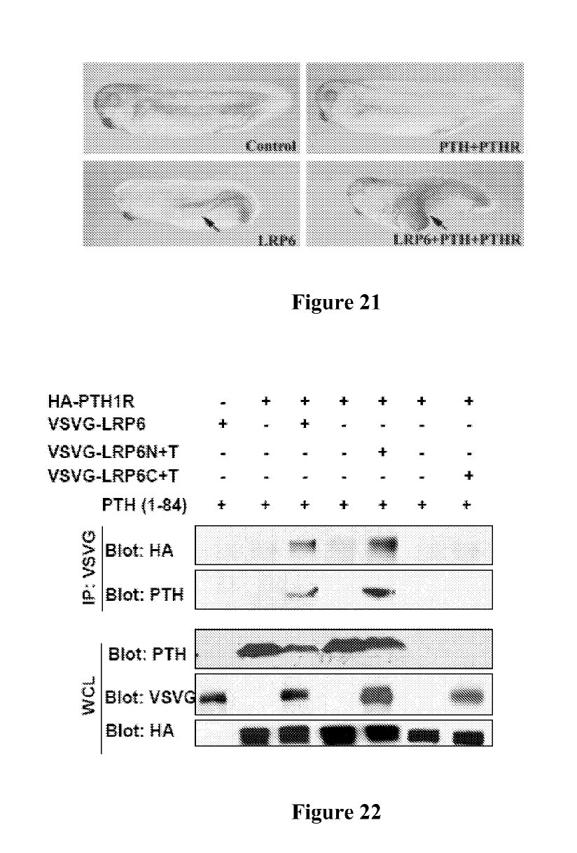

The possibility that LRP6 forms a ternary complex with PTH and PTH1R as it does with Wnt and Fz was then examined. Immunoprecipitation (IP) with antibodies to LRP6 from lysates of PTH-treated UMR-106 cells indicated that PTH1R formed a complex with endogenous LRP6 in response to PTH in a time-dependent manner (FIG. 14). Unlike LRP6, PTH did not enhance the binding of LRP5 to PTH1R although there is detectable binding in the absence of PTH (FIG. 15). The presence of PTH ligand in the LRP6-PTH1R complex was also indicated by co-IP. The PTH ligand was immunoprecipitated by LRP6 only when both LRP6 and PTH1R were present (FIG. 16). Further evidence for the PTH-PTH1R-LRP6 complex formation was obtained from PTH-induced close association of PTH1R with LRP6 in cells by photo bleaching-based fluorescence resonance energy transfer (FRET) (FIGS. 17-19). As shown in FIGS. 18 and 19, PTH led to increased FRET efficiency between CFP-PTH1R and YFP-LRP6, but did not enhance the FRET efficiency in either YFP-LRP6 and CFP-BMPRII, BMP type II coreceptor (Cao and Chen, Gene 357:1-8 (2005)), or between CFP-PTH1R and YFP-mLRP4T100, another member of the low-density lipoprotein-related proteins family (Li et al., J. Biol. Chem. 275:17187-94 (2000)). Thus, LRP6 specifically interacts with PTH1R upon PTH stimulation. The association of PTH1R with LRP6 is also supported by analysis of the model of LRP6-mediated secondary axis induction in Xenopus, in which PTH enhanced LRP6-induced secondary axis induction (FIGS. 20 and 21).

### Example 3

### Extracellular Domain of LRP6 Interacts with PTH1R

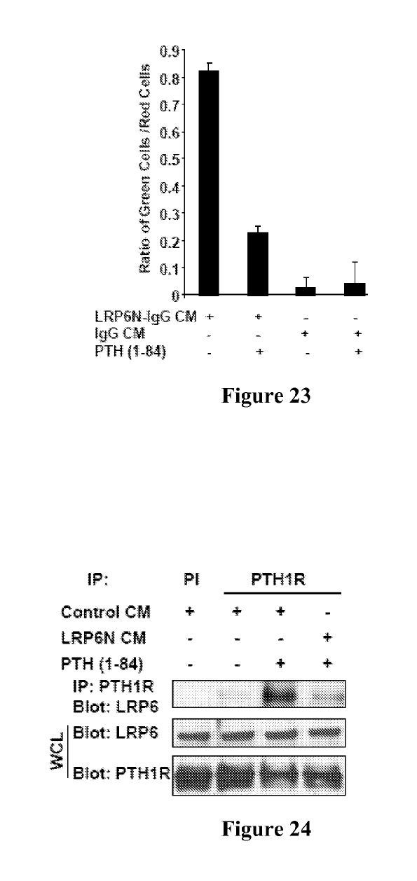

To confirm and extend the studies of the LRP6 and PTH1R complex formation, the region of LRP6 required for its interaction with PTH1R was mapped. PTH1R was co-expressed in cells with LRP6, a truncated LRP6 containing the extracellular and transmembrane domains (LRP6N+T), or the transmembrane and intracellular domains (LRP6T+C) for IP assay. Binding of LRP6T+C to PTH1R could barely be detected, but the LRP6N+T associated with PTH1R as effectively as did full-length LRP6 (FIG. 22). The presence of PTH in the LRP6N+T/PTH1R complex further suggested the formation of a ternary complex. Moreover, PTH-induced direct interaction of LRP6N with PTH1R on cell surface was confirmed in an immunofluorescence colocalization assay. Immun-colocalization of LRP6N-IgG with PTH1R on cell surface was increased from 22.8% to 82.3% with addition of PTH ligand whereas binding of IgG to PTH1R was barely detected (FIG. 23).

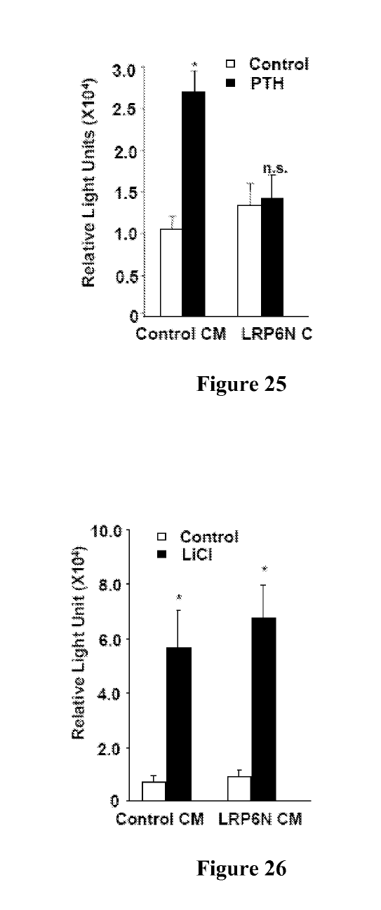

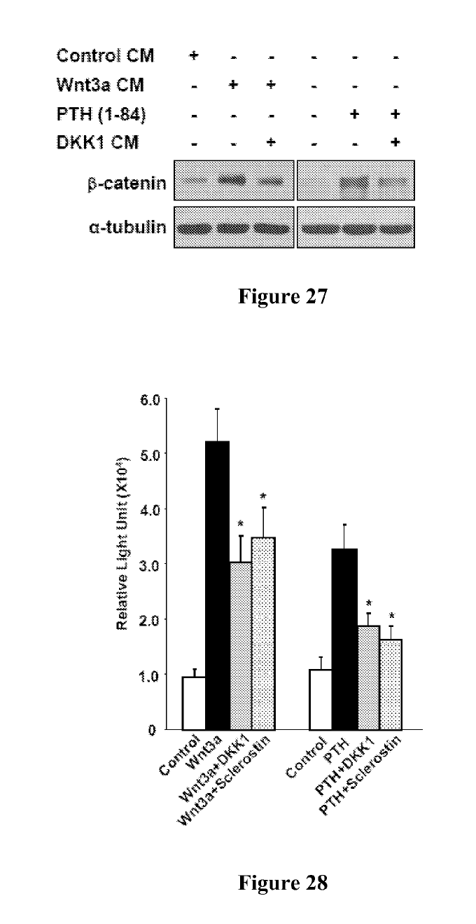

Whether LRP6N acts as a dominant-negative in PTH signaling through LRP6 was then examined. LRP6N blocked the PTH-induced association of endogenous LRP6 with PTH1R (FIG. 24). LRP6N inhibited PTH-, but not LiCl-induced TCF transcriptional activity (FIGS. 25 and 26). LiCl directly inhibits GSK3 kinase in the cytoplasm in the stabilization of β-catenin (Stambolic et al., Curr. Biol. 6:1664-8 (1996)), indicating that LRP6N acts upstream of GSK3 and funtions as a dominant-negative in the PTH-activated β-catenin signaling via binding to cell surface PTH1R. Furthermore, secreted proteins DKK1 and sclerostin, also binding to LRP6 at the extracellular domain (Mao et al., Nature 411:321-5 (2001); Li et al., J. Biol. Chem. 280:19883-7 (2005); and Semenov et al., J. Biol. Chem. 280:26770-5 (2005)), disrupted PTH-induced β-catenin accumulation in the cytoplasm (FIG. 27) and TCF/LEF luciferase activity (FIG. 28). Thus, PTH-induced recruitment of LRP6 through its extracellular domain is essential in activation of β-catenin signaling pathway.

### Example 4

### PTH Induces Phosphorylation of LRP6 and Axin Recruitment in Osteoblasts

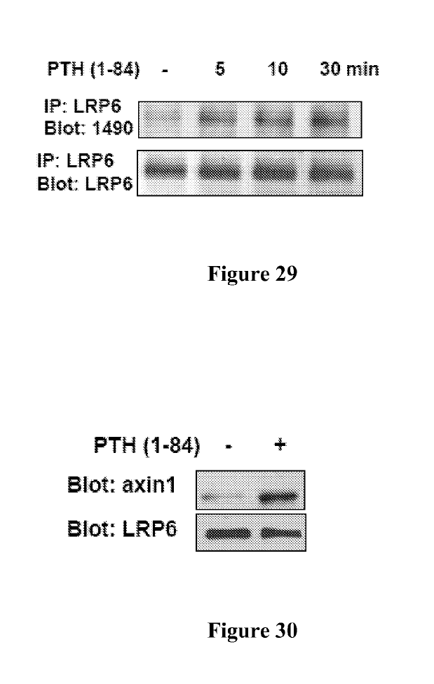

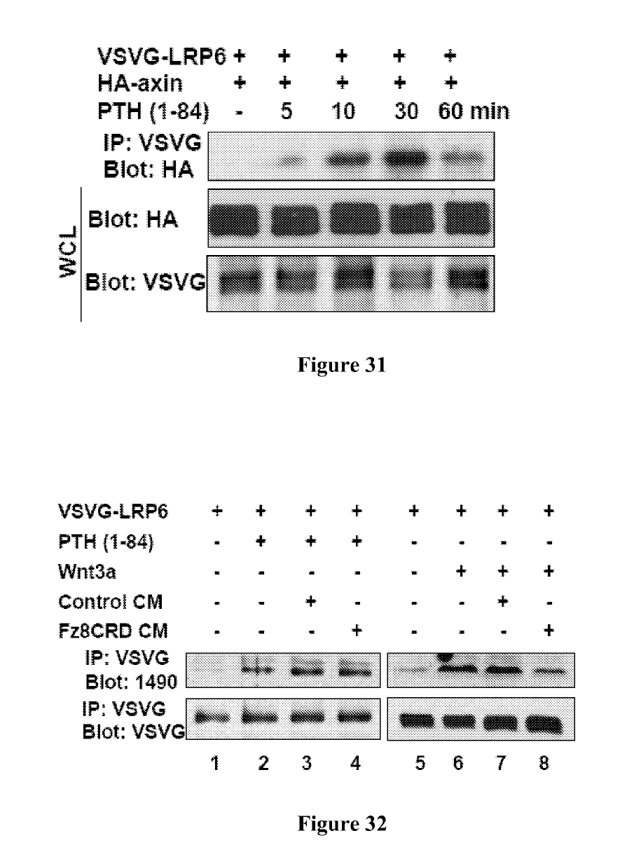

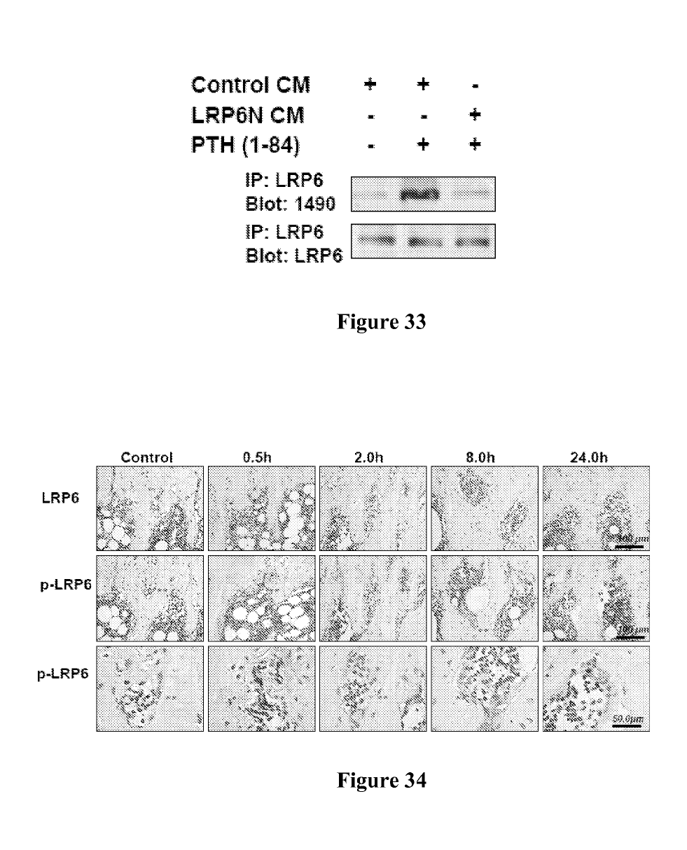

As phosphorylation of LRP6 at the PPPSP motifs plays a crucial role in activating downstream β-catenin signaling by Wnt, whether PTH induces phosphorylation of LRP6 at PPPSP motifs (Mao et al., Mol. Cell. 7:801-9 (2001); Tamai et al., Mol. Cell. 13:149-56 (2004); Davidson et al., Nature 438:867-72 (2005); Zeng et al., Nature 438:873-7 (2005)). Immunoprecipitated LRP6 from extracts of PTH-treated UMR-106 cells were monitored for their phosphorylation, by western blotting, with an antibody that recognizes phosphorylated PPPSP motifs (Ab1490) (Tamai et al., Mol. Cell. 13:149-56 (2004)). PTH rapidly induced the phosphorylation of LRP6 at the PPPSP motifs (FIG. 29). Phosphorylation of PPPSP motifs is required for axin recruitment from cytoplasm to LRP6 at cell membrane. PTH also increased axin1 level on cell membrane detected by cell fractionation assay in primary preosteoblasts (FIG. 30). Consistently, PTH rapidly increased in the binding of axin to LRP6 by co-IP assays (FIG. 31). Fz8CRD, a competitive inhibitor of the Wnt receptor Fz (Hsieh et al., Proc. Natl. Acad. Sci. USA 96:3546-55 (1999)), was used to exclude the possibility that these PTH effects are mediated through promotion of Wnts production or sensitization of Wnt-stimulated signaling. Fz8CRD inhibited Wnt3a-induced phosphorylation of LRP6 (FIG. 32, lane 8), but did not inhibit the effect of PTH (FIG. 33, lane 4). In contrast, LRP6N blocked PTH-stimulated LRP6 phosphorylation (FIG. 33).

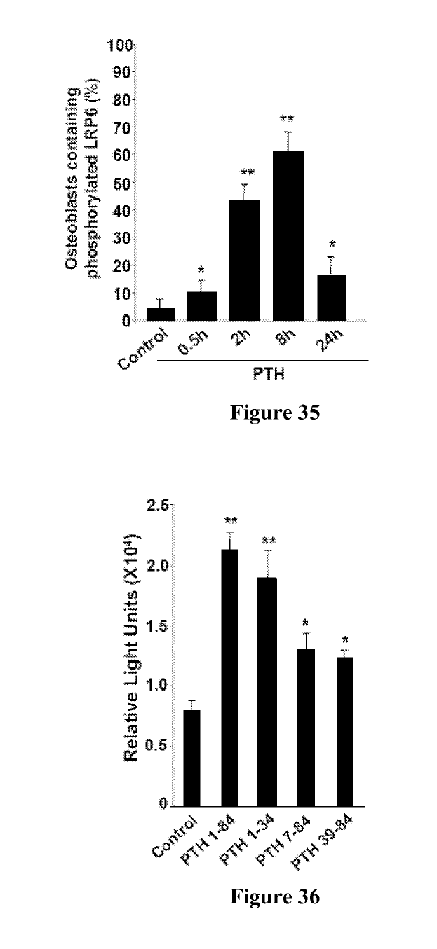

Because the levels of β-catenin were increased in osteoblasts of rats with injection of a single dose of PTH (FIGS. 6 and 7), it was tested whether the amounts of phosphorylated LRP6 were enhanced in osteoblasts from the same tissue. Immunostaining with an antibody specific for the phosphorylated PPPSP demonstrated that PTH-stimulated phosphorylation of LRP6 in preosteoblasts or osteoblasts at the surface of trabecular bone (FIG. 34, second and third rows), whereas the amount of total LRP6 protein remained unchanged (FIG. 34, first row). The temporal pattern of phosphorylation of the PPPSP motifs was similar to that of β-catenin (compare FIG. 6 and FIG. 34 second and third rows; and FIG. 7 and FIG. 35). Thus, PTH increases the abundance of β-catenin in osteoblasts in vivo through phosphorylation of LRP6.

### Example 5

### PKA is Required in PTH-, but not in Wnt-Activated LRP6-β-Catenin Signaling

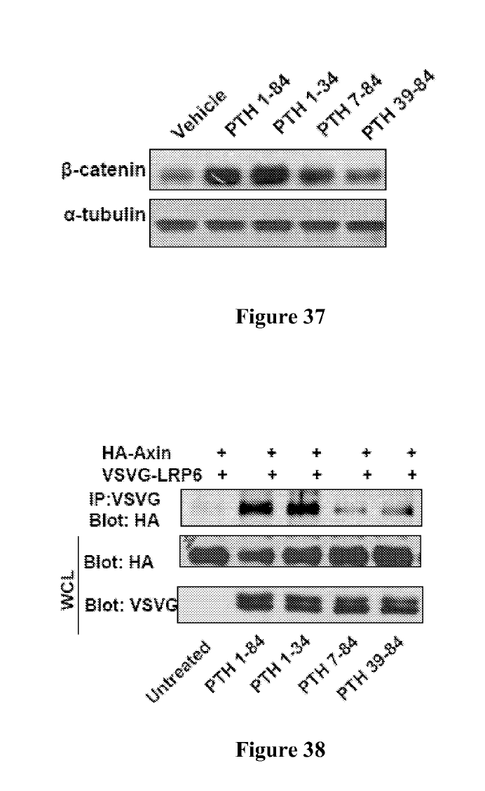

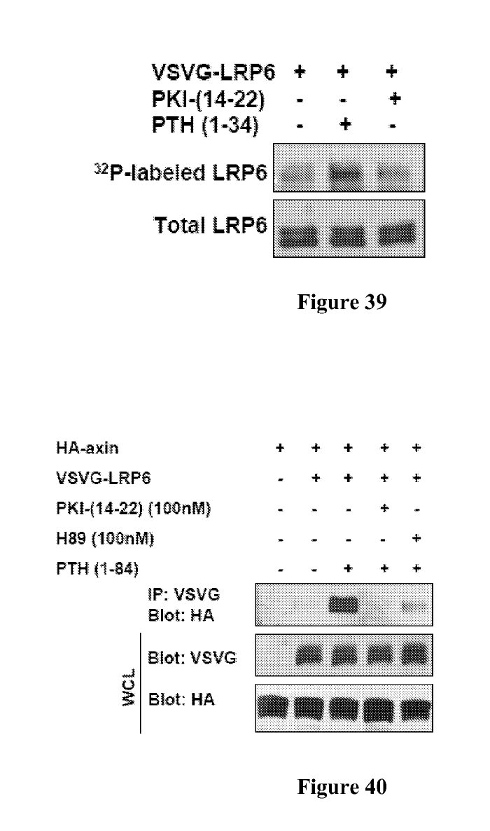

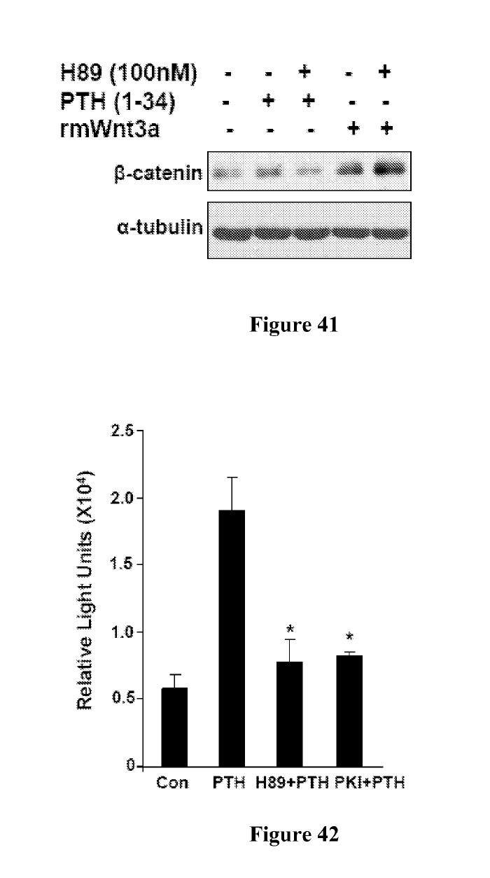

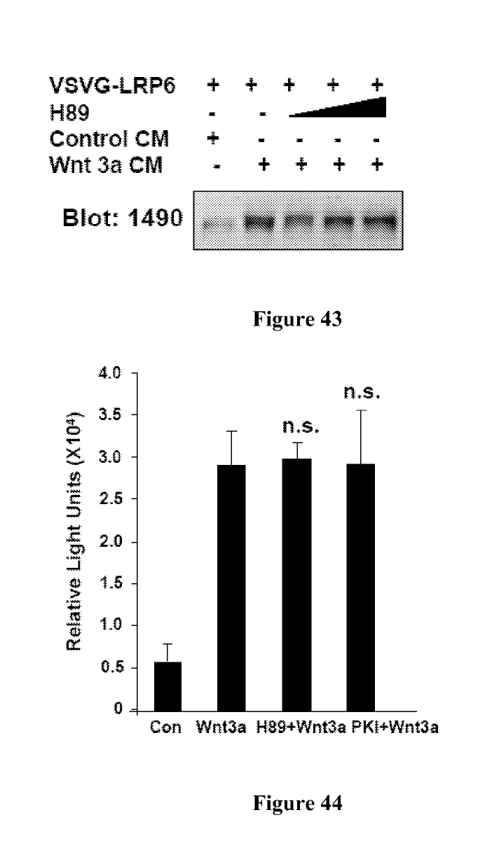

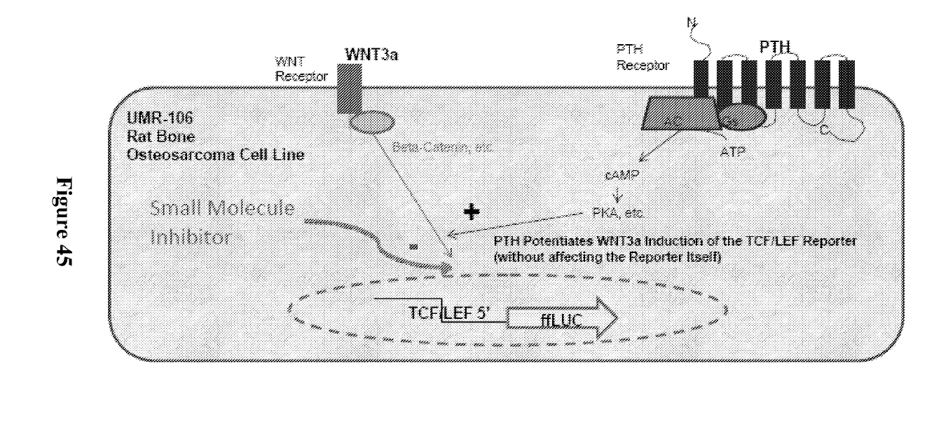

As the activation of β-catenin signaling by PTH in osteoblasts seems to be independent of Wnt, the mechanism responsible for the PTH effects was investigated. PTH activates cAMP-dependent PKA, which is sufficient for initiation of signals mediating PTH action in osteoblasts. Whether PKA participates in PTH-activated LRP6-β-catenin signaling was assessed. Binding of intact PTH (1-84) or PTH (1-34) to PTH1R activates PKA. However, the native C-terminal fragments of PTH bind PTH1R but do not activate PKA (Murray et al., Endocrine Rev. 26:78-113 (2005); Kronenberg et al., Recent Prog. Horm. Res. 53:283-301 (1998); Gensure et al., Biochem. Biophys. Res. Commun. 328:666-78 (2005)). The C-terminal fragments of PTH (7-84) and PTH (39-84) were much less effective than PTH (1-84) in activating β-catenin signaling (FIG. 36), altering the stability of β-catenin (FIG. 37), and inducing axin-LRP6 binding (FIG. 38). The minimum effects induced by PTH (7-84) and PTH (39-84) (FIGS. 36-38) may be mediated by other signaling components than PKA as the unrelated peptide of the similar length did not exert such effect. PKI (14-22), a specific inhibitor of PKA-directed phosphorylation, inhibited PTH-induced LRP6 phosphorylation (FIG. 39). Moreover, the PKA inhibitors, PKI (14-22) and H89 reduced the binding of axin to LRP6 (FIG. 40), β-catenin stabilization (FIG. 41 lane 3), and β-catenin-dependent transcription activity (FIG. 42), further indicating that PKA activity is essential for PTH-activated LRP6-β-catenin signaling. However, H89 did not affect Wnt3a-stimulated LRP6 phosphorylation (FIG. 43), β-catenin stabilization (FIG. 41, lane 5), and β-catenin-dependent transcription activity (FIG. 44).

### Example 6

### High-Throughput Screen to Determine PTH Agonists and Antagonists

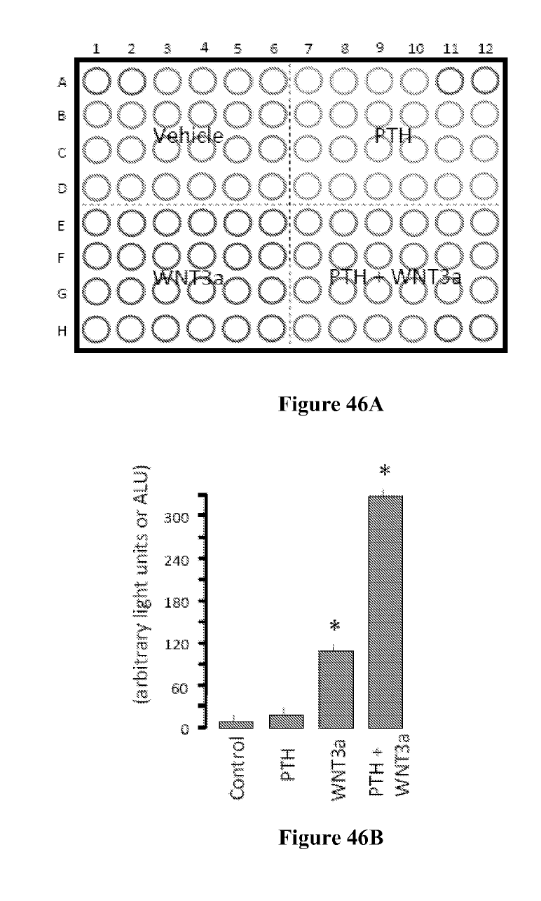

To determine agonists and antagonists of the additive signaling of the PTH receptor and the WNT receptor, (as well as any co-receptors) in bone cells, a high-throughput screen is designed. FIG. 45 shows a schematic of the high-throughput screen. An osteosarcoma cell line is transiently transfected with a TCF/LEF luciferase reporter. The cells are treated with WNT3a and PTH and luciferase activity is determined (FIG. 46). PTH treatment alone does not stimulate the TCF/LEF luciferase reporter (FIG. 46B). While WNT3a treatment stimulates the luciferase reporter, PTH and WNT3a treatment acts synergistically to stimulate luciferase activity (FIG. 46B), demonstrating that PTH potentiates WNT stimulation of the reporter.

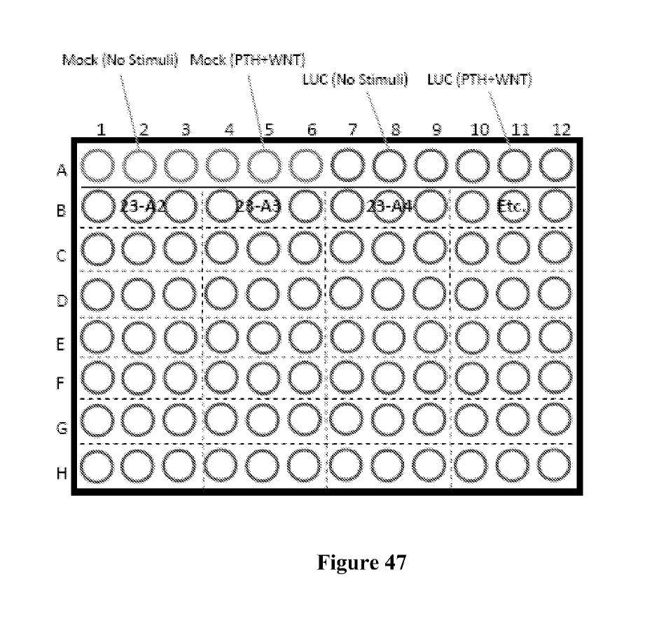

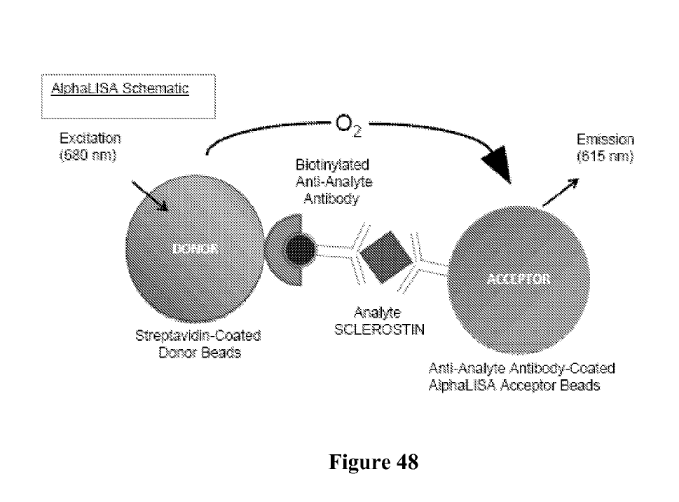

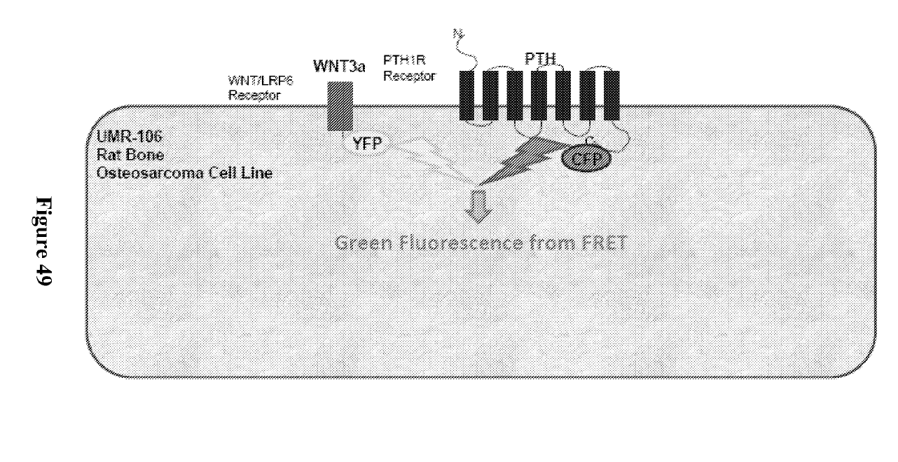

As PTH and WNT3a treatment stimulated luciferase reporter activity, a high-throughput assay is performed to test for small molecules that inhibit or enhance the synergistic effect of PTH and WNT3a treatment in osteosarcoma cells. FIG. 47 shows a schematic of a 96-well plate with controls and experimental wells depicted. Wells A1-A3 are mock transfected without stimuli, wells A4-A6 are mock transfected and stimulated with PTH and WNT, wells A7-A9 are transiently transfected with the luciferase reporter, and wells A10-A12 are transiently transfected with the luciferase reporter and stimulated with PTH and WNT. Wells B1-B3, B4-B6, B7-B9, B10-B12, C1-3, C4-C6. C7-C9, C10-C12, etc are treated with small molecules to be tested. The supernatant is harvested and an AlphaLISA® assay is performed to determine the amount of secreted osteosclerostin (FIG. 48). Cell lysates are made and expression of the luciferase reporter is assayed to determine if the small molecule is a PTH agonist or antagonist. Small molecules identified in the screen are further tested using the FRET assay shown in FIG. 49. The FRET assay determines whether the small molecules inhibit or stimulate the interaction between WNT/LRP6 and PTH1R at the level of the receptors at or near the cell membrane.

1-42. (canceled)

43. A method of screening for an agent that is a parathyroid hormone (PTH) agonist, the method comprising:
(a) contacting a cell with a parathyroid hormone (PTH) polypeptide or a receptor-binding fragment thereof, a WNT polypeptide or fragment thereof, and the agent to be screened, wherein the cell comprises a parathyroid hormone 1 receptor (PTH1R) and a nucleotide sequence encoding a reporter protein operably linked to an inducible promoter, wherein the inducible promoter is activated by PTH1R; and
(b) determining a level of reporter protein expression, an increased level of reporter protein expression as compared to a control indicating the agent is a PTH agonist.

44. The method of claim 43, wherein the WNT polypeptide or fragment thereof comprises a WNT3a polypeptide or fragment thereof.

45. (canceled)

46. (canceled)

47. The method of claim 43, wherein the inducible promoter comprises at least one T-cell factor/lymphoid enhancer factor (TCF/LEF) binding site.

48. (canceled)

49. The method of claim 43, wherein the cell is an osteosarcoma cell.

50. The method of claim 49, wherein the osteosarcoma cell is an UMR-106 cell.

51. (canceled)

52. A method of screening for an agent that is a parathyroid hormone (PTH) antagonist, the method comprising:
(a) contacting a cell with parathyroid hormone (PTH) polypeptide or a receptor-binding fragment thereof, a WNT polypeptide or fragment thereof, and the agent to be screened, wherein the cell comprises a parathyroid hormone 1 receptor (PTH1R) and a nucleotide sequence encoding a reporter protein operably linked to an inducible promoter, wherein the inducible promoter is activated by PTH1R; and
(b) determining a level of reporter protein expression, a decreased level of reporter protein expression as compared to a control indicating the agent is a PTH antagonist.

53. The method of claim 52, wherein the WNT polypeptide or fragment thereof comprises a WNT3a polypeptide or fragment thereof.

54. (canceled)

55. (canceled)

56. The method of claim 52, wherein the inducible promoter comprises at least one T-cell factor/lymphoid enhancer factor (TCF/LEF) binding site.

57. (canceled)

58. The method of claim 52, wherein the cell is an osteosarcoma cell.

59. The method of claim 58, wherein the osteosarcoma cell is an UMR-106 cell.

60. (canceled)

61. A method of screening for an agent that is a parathyroid hormone (PTH) agonist, the method comprising:
(a) contacting a cell with a parathyroid hormone (PTH) polypeptide or a receptor-binding fragment thereof, a WNT polypeptide or fragment thereof, and the agent to be screened, wherein the cell comprises a parathyroid hormone 1 receptor (PTH1R); and
(b) determining a level of secreted osteosclerostin polypeptide, an increased level of osteosclerostin as compared to a control indicating that the agent is a PTH agonist.

62. (canceled)

63. The method of claim 61, wherein the WNT polypeptide or fragment thereof comprises a WNT3a polypeptide or fragment thereof.

64. The method of claim 61, wherein the cell is an osteosarcoma cell.

65. The method of claim 64, wherein the osteosarcoma cell is an UMR-106 cell.

66. (canceled)

67. A method of screening for an agent that is a parathyroid hormone (PTH) antagonist, the method comprising:
(a) contacting a cell with a parathyroid hormone (PTH) polypeptide or a receptor-binding fragment thereof, a WNT polypeptide or fragment thereof, and the agent to be screened, wherein the cell comprises a parathyroid hormone 1 receptor (PTH1R); and
(b) determining a level of secreted osteosclerostin polypeptide, a decreased level of osteosclerostin as compared to a control indicating that the agent is a PTH antagonist.

68. (canceled)

69. The method of claim 67, wherein the WNT polypeptide or fragment thereof comprises a WNT3a polypeptide or fragment thereof.

70. The method of claim 67, wherein the cell is an osteosarcoma cell.

71. The method of claim 70, wherein the osteosarcoma cell is a UMR-106 cell.

72. (canceled)


SEQUENCE LISTING (6 sequences)

SEQ ID NO: 1 - DNA, 20 bp, Artificial Sequence
  Synthetic Oligonucleotide
gggtgtgaaccacgagaaat

SEQ ID NO: 2 - DNA, 20 bp, Artificial sequence
  Synthetic Oligonucleotide
ccttccacaatgccaaagtt

SEQ ID NO: 3 - DNA, 20 bp, Artificial sequence
  Synthetic Oligonucleotide
cttggtgcacacctagcaga

SEQ ID NO: 4 - DNA, 20 bp, Artificial sequence
  Synthetic Oligonucleotide
ctccctcatgtgttgtccct

SEQ ID NO: 5 - DNA, 20 bp, Artificial sequence
  Synthetic Oligonucleotide
ccaagatctctaacatgacg

SEQ ID NO: 6 - DNA, 20 bp, Artificial Sequence
  Synthetic Oligonucleotide
caccatcagctgaagatagt